(19) United States
(12) Patent Application Publication
(10) Pub. No.: US 2025/0177070 A1
(43) Pub. Date: Jun. 5, 2025

(54) DRIVE DEVICE, SURGICAL TOOL DEVICE, ARM DEVICE, AND MASTER-SLAVE SYSTEM

(71) Applicant: SONY GROUP CORPORATION, TOKYO (JP)

(72) Inventor: KAZUHITO WAKANA, TOKYO (JP)

(21) Appl. No.: 18/844,091

(22) PCT Filed: Jan. 23, 2023
(86) PCT No.: PCT/JP2023/001845

§ 371 Date: Sep. 5, 2024

(30) Foreign Application Priority Data
Mar. 15, 2022 (JP) 2022-040315

(51) Int. Cl. A61B 34/35; A61B 34/30

(52) U.S. Cl. CPC A61B 34/35; A61B 2034/305

(57) ABSTRACT

A drive device that drives an end effector is provided.

The drive device includes: a capstan; a cable wound around the capstan; and a rod that has one degree of freedom in linear motion, and performs a linear motion by rotation of the capstan, the cable being connected to the rod. In the drive device, an end effector is driven by the linear motion of the rod. The capstan assembly includes a reaction force applying unit that applies a reaction force to rotate the first capstan and the second capstan in directions opposite to each other between the first capstan and the second capstan, and applies a pre-tension force to cables by winding the cables around the first capstan and the second capstan by rotation caused by the reaction force.

### TECHNICAL FIELD

The technology disclosed in the present specification (hereinafter referred to as “the present disclosure”) relates to a drive device that drives an end effector, a surgical tool device including a surgical tool unit using a surgical tool for surgery as an end effector and a drive unit, an arm device that supports the surgical tool device, and a master-slave system that remotely operates the arm device.

### BACKGROUND ART

For example, a surgical manipulator that is used in the medical field normally has a configuration in which an end effector including a medical instrument such as a surgical tool is provided at the distal end, and the end effector is driven by a rotative force of a motor disposed on the base side. However, a mechanism for converting the rotative force of the motor into a linear motion force is required, and there are problems in that the structure of a device for a rotational-to-linear motion converting device becomes complicated, and the device becomes larger in size.

For example, a surgical device in which a surgical instrument is coupled to a motor in a surgical instrument manipulator assembly via a mechanical interface has been suggested (see Patent Document 1). This surgical device adopts a mechanism that converts rotation of the motor into a linear motion with a ball screw, to impart a linear motion to a cable for operating the surgical instrument. When a ball screw is used, there are problems in that the surgical device is elongated in the axial direction of the ball screw, a backlash occurs, and backdrivability becomes lower.

### CITATION LIST

### Patent Document

Patent Document 1: Japanese Patent Application Laid-Open No. 2020-151524
Patent Document 2: Japanese Patent Application Laid-Open No. 2021-41037, paragraph 0030, paragraph 0055, and FIG. 5

### SUMMARY OF THE INVENTION

### Problems to be Solved by the Invention

The present disclosure aims to provide a drive device that drives an end effector by converting a rotative force of a motor into a linear motion force, a surgical tool device including a surgical tool unit using a surgical tool for surgery as an end effector and a drive unit, an arm device that supports the surgical tool device, and a master-slave system that remotely operates the arm device.

### Solutions to Problems

The present disclosure is made in view of the above problems, and a first aspect thereof is a drive device that includes
a rod that has one degree of freedom in linear motion, and performs a linear motion by rotation of the capstan, the cable being connected to the rod,
in which a replaceably attached end effector is driven by the linear motion of the rod.

The cable includes a pair of cables that is wound around the capstan in directions opposite to each other, and is connected to the rod in forward and backward directions. Further, the rod moves forward and backward in accordance with a direction of rotation of the capstan.

Furthermore, the capstan assembly includes a reaction force applying unit that applies a reaction force to rotate the first capstan and the second capstan in directions opposite to each other between the first capstan and the second capstan, and applies a pre-tension force to the pair of cables by winding the pair of cables around the first capstan and the second capstan by rotation caused by the reaction force.

Further, a second aspect of the present disclosure is
a surgical tool device that includes:
a drive unit including: a motor; a capstan attached to an input shaft or an output shaft of the motor; a cable wound around the capstan; and a rod that linearly moves by rotation of the motor, the cable being connected to the rod; and
a surgical tool unit that is replaceably attached to the drive unit, and drives a surgical tool with a linear motion force transmitted via the rod.

Furthermore, a third aspect of the present disclosure is
an arm device that includes:
a surgical tool device including: a drive unit including: a motor; a capstan attached to an input shaft or an output shaft of the motor; a cable wound around the capstan; and a rod that linearly moves by rotation of the motor, the cable being connected to the rod; and a surgical tool unit that is replaceably attached to the drive unit, and drives a surgical tool with a linear motion force transmitted via the rod; and
an arm of an articulated link structure that supports the surgical tool device.

Further, a fourth aspect of the present disclosure is
a master-slave system that includes:
a slave device including: a surgical tool device including: a drive unit including: a motor; a capstan attached to an input shaft or an output shaft of the motor; a cable wound around the capstan; and a rod that linearly moves by rotation of the motor, the cable being connected to the rod; and a surgical tool unit that is replaceably attached to the drive unit, and drives a surgical tool with a linear motion force transmitted via the rod; and an arm of an articulated link structure that supports the surgical tool device; and
a master device that operates the surgical tool device and the arm.

It should be noted that the term “system” used herein means a logical assembly of multiple devices (or functional modules that implement specific functions), and it does not matter whether or not each of the devices or functional modules is in a single housing. That is, a device including multiple components or functional modules, and an assembly of multiple devices both correspond to a “system”.

### Effects of the Invention

According to the present disclosure, it is possible to provide a drive device that converts a rotative force of a motor into a linear motion force, transmits the linear motion force to a replaceably attached end effector, and has a high backdrivability without any backlash, a surgical tool device that includes a replaceable surgical tool unit having a surgical tool for surgery as an end effector and a drive unit, an arm device that supports the surgical tool device, and a master-slave system that remotely operates the arm device.

Note that the effects described in the present specification are merely examples, and the effects to be brought about by the present disclosure are not limited to them. Furthermore, there are cases where the present disclosure further provides some other effects, in addition to the effects described above.

Still other objects, features, and advantages of the present disclosure will become apparent from a more detailed description based on embodiments as described later and the accompanying drawings.

### BRIEF DESCRIPTION OF DRAWINGS

FIG. 1 is a view illustrating a surgical tool device 100 in a state where a surgical tool unit 101 is attached to a drive unit 103.

FIG. 2 is a view illustrating the surgical tool device 100 in a state where the surgical tool unit 101 is separated from the drive unit 103.

FIG. 3 is a view illustrating a state in which the drive unit 103 in a state in which the surgical tool unit 101 is attached is further mounted on an arm device 300.

FIG. 4 is a view illustrating an internal configuration with a cross-sectional view of the surgical tool unit 101.

FIG. 5 is a view illustrating an internal configuration with a cross-sectional view of an adapter unit 102 and the drive unit 103 taken along a plane including a longitudinal direction.

FIG. 6 is a view illustrating an image in which the surgical tool unit 101 is attached to the drive unit.

FIG. 7 is a view illustrating a specific configuration of the surgical tool unit 101.

FIG. 8 is a view illustrating a cross-sectional configuration of the surgical tool unit 101 illustrated in FIG. 7.

FIG. 9 is a perspective view of an inner base 703.

FIG. 10 is a view illustrating a cross-section of the surgical tool unit 101 taken along a plane 750 orthogonal to the longitudinal direction.

FIG. 11 is a view illustrating a perspective view of the root side of the surgical tool unit 101 taken along the plane 750.

FIG. 12 is an exploded view of the surgical tool unit 101 exploded in the longitudinal direction.

FIG. 13 is a perspective view 13 of a rod 713.

FIG. 14 is a diagram illustrating a state in which four sets of a motor and a rotational-to-linear motion converting device are provided for the drive unit 103.

FIG. 15A is a perspective view of a side surface of one set of a motor and a rotational-to-linear motion converting device.

FIG. 15B is a perspective view of a side surface of one set of a motor and a rotational-to-linear motion converting device, as viewed from the opposite side from that in FIG. 15A.

FIG. 16 is an enlarged view and an exploded view of the vicinity of motor capstans 1521 and 1522.

FIG. 17 is cross-sectional views of the surgical tool device 100.

FIG. 18A is a cross-sectional view of the adapter unit 102.

FIG. 18B is a perspective view of the adapter unit 102.

FIG. 19 is an exploded view of the adapter unit 102 exploded in the longitudinal direction.

FIG. 20 is an exploded view of the adapter unit 102 exploded in the longitudinal direction.

FIG. 21 is an enlarged view of the vicinity of the root of the surgical tool unit 101 attached to the adapter unit 102.

FIG. 22 is front views illustrating respective states before attachment, in a middle of the attachment, and at the position of the attachment of the surgical tool unit 101 to the adapter unit 102.

FIG. 23 is views illustrating a procedure for attaching the surgical tool unit 101 to the adapter unit 102 (before attachment).

FIG. 24 is views illustrating a procedure for attaching the surgical tool unit 101 to the adapter unit 102 (in the middle of the attachment).

FIG. 25 is views illustrating a procedure for attaching the surgical tool unit 101 to the adapter unit 102 (in the middle of the attachment).

FIG. 26 is views illustrating a procedure for attaching the surgical tool unit 101 to the adapter unit 102 (in the middle of attachment).

FIG. 27 is views illustrating a procedure for attaching the surgical tool unit 101 to the adapter unit 102 (at the time of completion of the attachment).

FIG. 28 is views illustrating a procedure for detaching the surgical tool unit 101 from the adapter unit 102 (before detachment).

FIG. 29 is views illustrating a procedure for detaching the surgical tool unit 101 from the adapter unit 102 (in the middle of the detachment).

FIG. 30 is views illustrating a procedure for detaching the surgical tool unit 101 from the adapter unit 102 (in the middle of the detachment).

FIG. 31 is views illustrating a procedure for detaching the surgical tool unit 101 from the adapter unit 102 (in the middle of the detachment).

FIG. 32 is views illustrating a procedure for detaching the surgical tool unit 101 from the adapter unit 102 (at the time of completion of the detachment).

FIG. 33 is a perspective view of forceps.

FIG. 34 is a top view of the forceps.

FIG. 35 is a side view of the forceps.

FIG. 36 is a diagram illustrating the degrees of freedom in the configuration of the forceps.

FIG. 37 is a diagram illustrating an example of the layout of motors M1 to M4 on the side of the drive unit 103.

FIG. 38 is a table illustrating input parameters for the drive unit 103 and output parameters on the side of the surgical tool unit 101.

FIG. 39 is views illustrating a series of operations in which the forceps at the distal end of the surgical tool unit 101 performs a yaw operation.

FIG. 40 is views illustrating a series of operations in which the forceps at the distal end of the surgical tool unit 101 performs a pitch operation.

FIG. 41 is views illustrating a series of operations in which the forceps at the distal end of the surgical tool unit 101 performs an opening/closing operation.

FIG. 42 is a view illustrating an example of the degrees of freedom in the configuration of the arm device 300.

FIG. 43 is views illustrating a series of operations in which the arm device 300 pans the surgical tool device 100.

FIG. 44 is views illustrating a series of operations in which the arm device 300 causes the surgical tool device 100 to tilt with respect to the main unit of the arm device 300.

FIG. 45 is views illustrating a series of operations in which the arm device 300 causes the surgical tool device 100 to tilt at the current position.

FIG. 46 is a diagram illustrating an example of the functional configuration of a master-slave system 4600.

### MODE FOR CARRYING OUT THE INVENTION

In the description below, the present disclosure will be explained in the following order with reference to the drawings.
A. Outline
B. Overall Configuration
C. Basic Configuration and Operation of a Surgical Tool Device
D. Detailed Configuration of a Surgical Tool Unit
E. Detailed Configuration of a Drive Unit
F. Downsizing of a Rotational-to-Linear Motion Converting Device
G. Attaching/Detaching Structure, Attaching Procedures, and Detaching Procedures for a Surgical Tool Unit
G-1. Attaching/Detaching Structure for a Surgical Tool Unit
G-2. Attaching Procedures for a Surgical Tool Unit
G-3. Detaching Procedures for a Surgical Tool Unit
H. Input-Output Relationship Between a Drive Unit and a Surgical Tool Unit
I. Arm Device
J. Master-Slave System

### A. Outline

The present disclosure relates to a drive unit that has an end effector unit interchangeably mounted thereto, and supplies a drive force for driving an end effector supported at a distal end of the end effector unit. In the present specification, embodiments in which the present disclosure is applied to the medical field will be mainly described. In such embodiments, an end effector is a surgical tool. Hereinafter, an end effector unit will be also referred to as a “surgical tool unit”, and will be also referred to as a “surgical tool device” in a state where the surgical tool unit is attached to a drive unit.

The surgical tool device is mounted on an arm device (also called a surgical robot or a surgical manipulator), for example, and is used for surgery. Meanwhile, the surgical tools are various kinds of medical instruments such as forceps, a pneumoperitoneum tube, an energy treatment tool, tweezers, and a retractor, for example. Therefore, an operation of preparing a plurality of types of surgical tool units including medical instruments of different types at the distal end, replacing the surgical tool unit attached to the drive unit as necessary during the surgery, and further, automatically replacing the surgical tool unit with a robot is conceivable.

If the drive unit can be manufactured small in size and light in weight, the entire arm device can be significantly made smaller in size and weight. If the surgical robot is downsized by reducing the size and weight of the arm device, a surgical robot can be adopted in a wide variety of operating rooms.

Further, for a robot to automatically change surgical tool units, it is desirable that the plurality of surgical tool units to be changed is installed on a mounting table such as a “surgical tool stand”. At the time of replacement, it is necessary to perform an action of greatly moving the arm device to the surgical stand to retrieve the surgical tool unit. If the arm device can be made smaller in size, the motion range of the arm device at a time of automatic surgical tool replacement becomes smaller. Thus, the risk of contact with the environment can be lowered, and the necessary work space can be made smaller. Furthermore, if the arm device is small in size and light in weight, the risk of the arm device destroying the other side when coming into contact with the environment or getting out of control is lowered, and safety can be enhanced.

Here, the configurations of the surgical tool device and the drive unit are discussed. In a case where the surgical tool at the distal end on the surgical tool unit side is driven directly by a rotational motion of a motor on the drive unit side, the structure of the drive unit can be simplified and downsized. In a case where the surgical tool is operated by a linear motion, on the other hand, a power conversion mechanism that converts a rotational motion of the motor on the drive unit side into a linear motion is required, and the structure of the surgical tool device becomes complicated and larger in size.

For example, Patent Document 1 discloses a surgical instrument that performs rotational-to-linear motion conversion using a ball screw. However, when a ball screw is used, there are problems in that the surgical device is elongated in the axial direction of the ball screw, a backlash occurs, and backdrivability is degraded. Also, if a rotational-to-linear motion converting device is mounted on the surgical tool unit side, the surgical tool unit as a replacement part becomes large and the surgical tool stand also becomes larger, and therefore, the motion range of the arm device at the time of automatic replacement becomes wider. Also, the cost of each surgical tool unit becomes higher.

In view of this, the present disclosure proposes a technique for equipping a drive unit with a rotational-to-linear motion converting device that is simple and compact, and operates without any backlash but with a high backdrivability.

With the use of the rotational-to-linear motion converting device that operates without any backlash but with a high backdrivability according to the present disclosure, it is easier to apply a surgical tool device to a surgical robot that performs delicate work. When backdrivability is high, an external force acting on the surgical tool unit (or the surgical tool at the distal end thereof) can be measured on the basis of the current value of the motor on the drive unit side.

Further, as the size and the weight of the rotational-to-linear motion converting device are reduced according to the present disclosure, the drive unit equipped with the rotational-to-linear motion converting device, and the entire arm device are made smaller in size and weight, and the rotational-to-linear motion converting device is readily applied to a wide variety of operating rooms. As the rotational-to-linear motion converting device is mounted on the drive unit side, instead of the surgical tool unit side, the cost of each surgical tool unit as a replacement part is prevented from becoming higher, and the surgical tool units can be easily made smaller in size and diameter. Since the surgical tool stand is also downsized and easily disposed near the surgical robot, the motion range of the arm device at a time of replacement is smaller, the risk of contact with the environment is lowered, and the required work space can be smaller.

### B. Overall Configuration

In this section B, an overall configuration of a surgical tool device including a surgical tool unit and a drive unit is described.

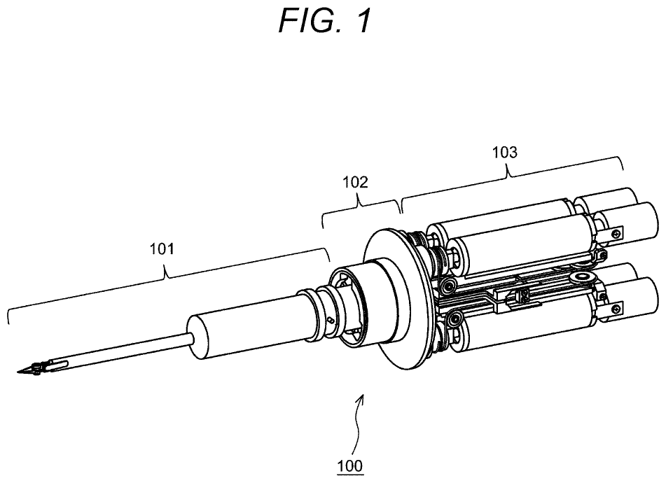

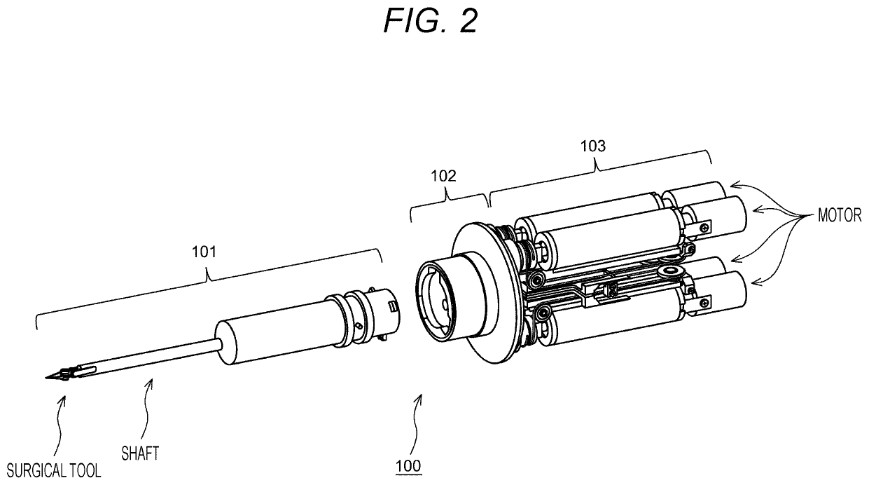

FIG. 1 illustrates a surgical tool device 100 in a state where a surgical tool unit 101 is attached to a drive unit 103. Further, FIG. 2 illustrates the surgical tool device 100 in a state where the surgical tool unit 101 is separated from the drive unit 103. The surgical tool device 100 includes the surgical tool unit 101, an adapter unit 102, and the drive unit 103 in this order from the distal end side. Note that, in actual surgery, it is necessary to separate the surgical tool unit 101 disposed in a clean region from an unclean region on the root side, and it is assumed that a drape (not illustrated) is provided and used between the surgical tool unit 101 and the adapter unit 102, or between the adapter unit 102 and the drive unit 103.

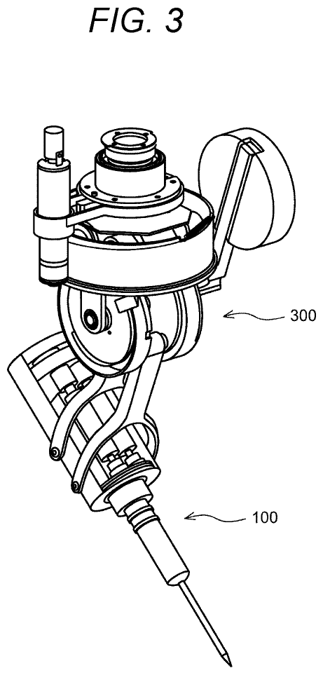

As illustrated in FIGS. 1 and 2, the adapter unit 102 for receiving (or inserting) the root side of the surgical tool unit 101 is attached to the tip end of the drive unit 103. Accordingly, it can be said that the surgical tool unit 101 is attached to the drive unit 103 via the adapter unit 102. Furthermore, FIG. 3 illustrates a state in which the surgical tool device 100 in the state where the surgical tool unit 101 is attached to the drive unit 103 is further mounted on an arm device 300. The arm device 300 can cause the surgical tool unit 101 attached to the drive unit 103 to perform a pan operation and a tilt operation, which will be described later.

The surgical tool unit 101 includes a surgical tool, and a hollow shaft that supports the surgical tool at the tip end (or the distal end). The surgical tools include various kinds of medical instruments such as forceps, a pneumoperitoneum tube, an energy treatment tool, tweezers, and a retractor, for example. In the description below, however, an embodiment specialized for forceps including a pair of jaws that opens and closes the surgical tool will be mainly described, for convenience sake.

In the present embodiment, the structure of forceps adopted as an end effector, which is a surgical tool, is briefly described. The forceps include a pair of jaws. Each of the jaws is rotatably supported about the opening/closing axis (or the yaw axis) near the tip end of the shaft, and a torsion spring that applies beforehand an opening force acting in a direction of opening (or in a direction where the jaws are separated from each other) about the opening/closing axis between the jaws is incorporated into the opening/closing axis (or the yaw axis). The forceps including the pair of jaws is then supported by the distal end of the shaft so as to be rotatable about a pitch axis (wrist) orthogonal to each of the opening/closing axis and the longitudinal axis of the shaft.

A cable (not illustrated in FIGS. 1 and 2) for transmitting a drive force generated by the drive unit 103 to the surgical tool at the distal end is inserted into the shaft of the surgical tool unit 101. In the case where the surgical tool is formed with a pair of jaws as described in the preceding paragraph, a total of four cables including one cable for pulling each of the jaws in a closing (or approaching the other jaw) direction about the opening/closing axis and one cable for rotating the entire forceps including the pair of jaws for each of forward and backward rotations about the pitch axis (wrist) are inserted into the shaft.

Furthermore, as described later, each cable inserted into the shaft is wound around a pulley on the root side of the surgical tool unit 101 (or the shaft), is folded back in the direction toward the distal end (or the tip end), and is then connected to a rod serving as a linear motion transmitting device (or the vicinity of the end is secured). A corresponding rod is required for each cable, and a total of four rods are arranged on the root side of the surgical tool unit 101. Each rod has only one degree of freedom to linearly move in the longitudinal direction of the surgical tool unit 101 (or the shaft). When the rod advances to the proximal side, the cable connected to the rod is pulled toward the root side to transmit the drive force.

As can be seen from FIGS. 1 and 2, the root side of the surgical tool unit 101 is attached to the tip end side of the drive unit 103 via the adapter unit 102. As long as each rod on the side of the surgical tool unit 101 can be attached so as to come right into contact with the corresponding rod on the side of the drive unit 103, the method and the structure for attaching the surgical tool unit 101 to the adapter unit 102 are not limited to any particular ones.

The drive unit 103 includes the number of sets of the motor that generates the drive force and the rotational-to-linear motion converting device that converts a rotational motion of the motor into a linear motion, the number corresponding to the number of cables on the side of the surgical tool unit 101. Furthermore, each motor includes an encoder and a brake. Each motor may have a planetary gear mechanism therein, but the present disclosure is not limited to this. As described above, since the surgical tool unit 101 includes four cables, the drive unit 103 is equipped with four sets of the motor and the rotational-to-linear motion converting device. Four cylindrical parts disposed on the proximal side of the drive unit 103 illustrated in FIGS. 1 and 2 are the respective motors. The rotational-to-linear motion converters provided for the respective motors are not illustrated in FIGS. 1 and 2.

### C. Basic Configuration and Operation of a Surgical Tool Device

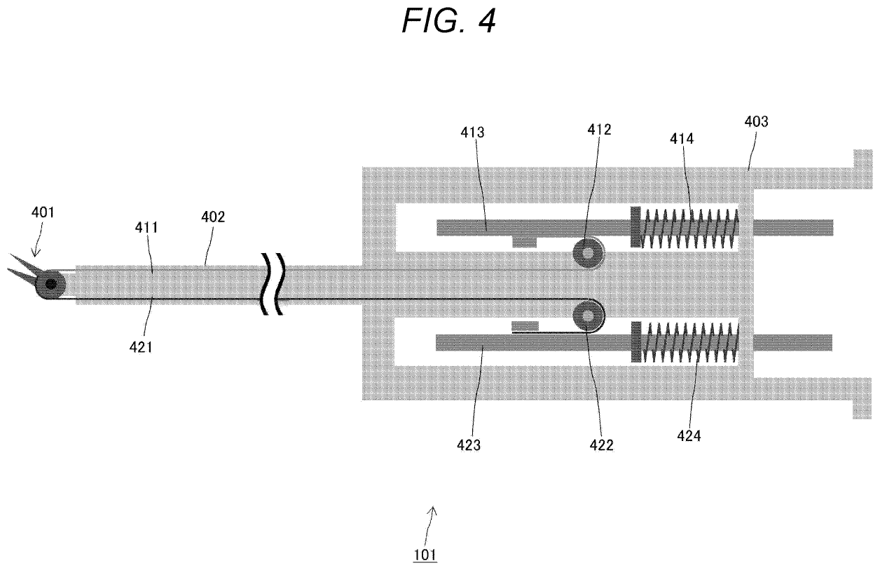

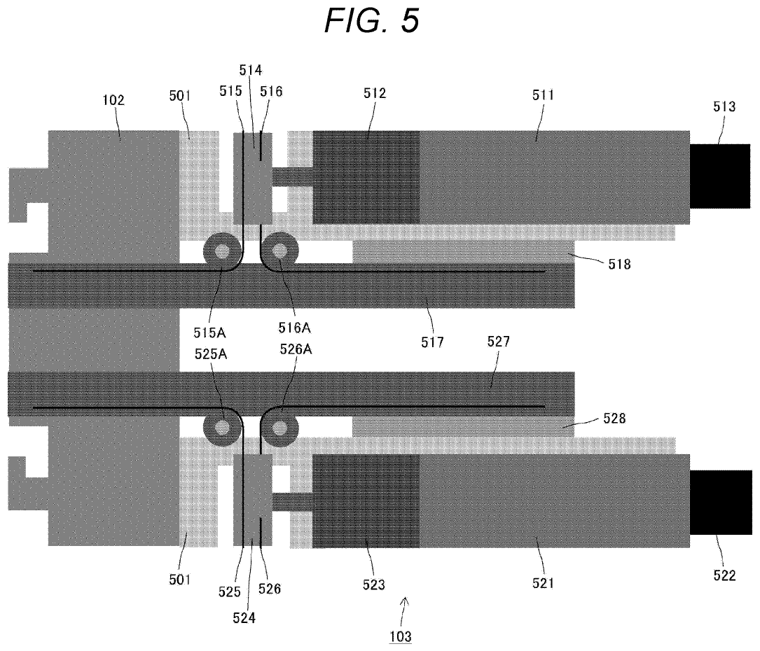

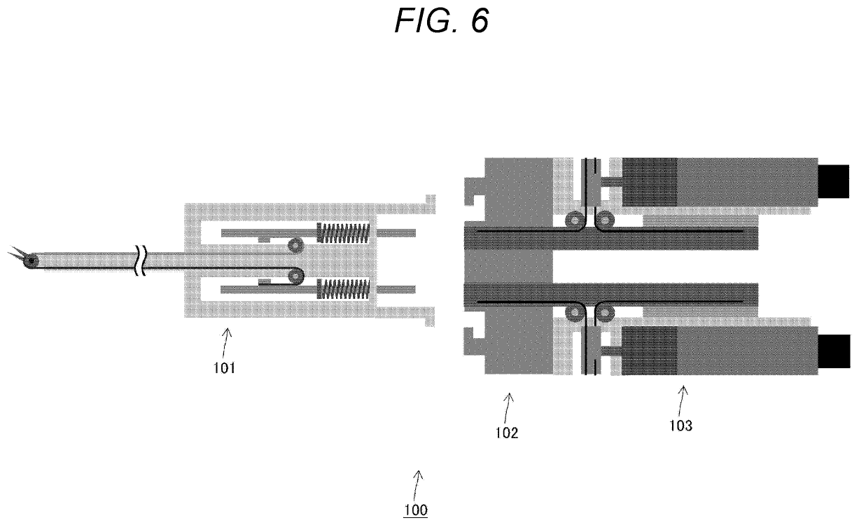

In this section C, the basic configuration and operation of the surgical tool device 100 are described. As described in the above section B, the surgical tool device 100 includes the surgical tool unit 101 and the drive unit 103, and the surgical tool unit 101 is attached to the drive unit 103 via the adapter unit 102 and is replaceable. FIG. 4 illustrates the internal configuration with a cross-sectional view of the surgical tool unit 101 taken along a plane including the longitudinal direction. FIG. 5 illustrates the internal configuration with a cross-sectional view of the adapter unit 102 and the drive unit 103 taken along a plane including the longitudinal direction. Note that an x- and y-axes are defined as illustrated in FIG. 4, for convenience sake. The x-axis corresponds to the longitudinal axis. Further, FIG. 6 illustrates an image in which the surgical tool unit 101 illustrated in FIG. 4, and the adapter unit 102 and the drive unit 103 illustrated in FIG. 5 are juxtaposed in the longitudinal direction, and the surgical tool unit 101 is attached to the drive unit 103.

First, the operating principles of a linear motion transmission mechanism mounted on the surgical tool unit 101 are described with reference to FIG. 4. The surgical tool unit 101 includes a surgical tool 401, a hollow shaft 402 that supports the surgical tool 401 at the tip end (or the distal end), and a surgical tool unit base 403 that supports the shaft 402 and is coupled to the adapter 102 on the root side. FIG. 4 illustrates the internal configuration of each of the shaft 402 and the surgical tool unit base 403 with the cross-sectional view of the surgical tool unit 101 taken along a plane including the longitudinal direction.

As described above, the surgical tool unit 101 includes the surgical tool 401 including a pair of jaws, and the total of four cables including two cables 411 and 421 that pull the respective jaws about the opening/closing axis, and two cables (not illustrated in FIG. 4) that rotate the surgical tool 401 (or the opening/closing axis of the jaws) forward and backward about the pitch axis (wrist). The surgical tool 401 has three degrees of freedom in opening and closing (or gripping) of each jaw, and respective rotations about the opening/closing axis (or the yaw axis) and the pitch axis of the forceps, for example, but details of this aspect are not described herein, for ease of explanation. Note that the number of cables necessary for driving the surgical tool 401 varies depending on the degree of freedom in the configuration of the surgical tool 401 and the like. FIG. 4 illustrates the two cables 411 and 421 and two linear motion transmitting devices corresponding to the respective cables 411 and 421, for simplification of the drawing. Furthermore, hereinafter, for convenience sake, explanation will be made, not imposing any particular restriction as to whether each of the cables 411 and 421 relates to any degree of freedom in the opening/closing (or gripping) of the surgical tool 401 (jaws) as the end effector, and rotations of the surgical tool 401 about the yaw axis and the pitch axis.

One end of the tip end side (or the distal side) of the cable 411 is connected to a capstan (not illustrated) related to any one of the degrees of freedom in the opening/closing (or gripping), pitch, and yaw of the surgical tool 401 (jaws) as an end effector. Furthermore, the other end of the cable 411 extending to the root side (or the proximal side) is inserted into the shaft 402, is then drawn into the surgical tool unit base 403, is wound around a pulley 412 on the root side of the surgical tool unit 101 (or the shaft 402), is folded back in the direction of the distal end (or the tip end), and is then connected to a rod 413 via a cable coupling portion (described below) (or the vicinity of the end is secured). The rod 413 is supported by the surgical tool unit base 403 so as to slide only with one degree of freedom in linear motion in the x-axis direction, which is the longitudinal direction. Further, the pulley 412 is an idler pulley, and is rotatably supported by the surgical tool unit base 403.

As will be described later with reference to FIG. 5, the rod 413 retreats and advances in the x-axis direction by the drive force transmitted from the side of the drive unit 103. When the rod 413 retreats in the x-axis direction (in other words, advances in a distal direction), the cable 411 connected to the rod 413 is then pulled, to transmit the drive force to the capstan related to any one of the degrees of freedom of the surgical tool 401 serving as an end effector. Thus, the rod 413 serves as a linear motion transmitting device.

This is because, when the rod 413 rotates about its own longitudinal axis, the cable 411 is wound around the rod 413, and fails to accurately drive the surgical tool 401 in accordance with the amount of linear motion of the rod 413 in the longitudinal direction. Therefore, the rod 413 is equipped with a rotation inhibiting device so as to inhibit rotation about the longitudinal axis and to operate only with the one degree of freedom in linear motion in the longitudinal direction. However, this aspect will be described later in detail.

Furthermore, a spring 414 that applies a force to push the rod 413 in the tip end direction is disposed on the root side of the rod 413 so that the cable 411 is not loosened even in a state where the surgical tool unit 401 is separated from the drive unit 403 to be independent. The rod 413 is inserted into the spring 414, and one end of the spring 414 is secured to the surgical tool unit 403 while the other end is secured to the rod 413. Therefore, when the rod 413 is pushed in the tip end direction by an elastic force of the spring 414, a pre-tension force is applied to the cable 411 folded back by the pulley 412 and coupled to the rod 413. Thus, the cable 411 is not loosened.

The cable 421 is similar to the cable 411. One end of the cable 421 on the tip end side (or the distal side) is connected to a capstan (not illustrated) related to any one of the degrees of freedom of the surgical tool 401 (jaws) as an end effector, and the other end of the cable 421 extending to the root side (or the proximal side) is inserted into the shaft 402, is then drawn into the surgical tool unit base 403, is wound around a pulley 422 on the root side of the surgical tool unit 101 (or the shaft 402), is folded back, and is then connected to a rod 423 via a cable coupling portion (described later) (or the vicinity of the end is secured). The rod 413 is supported by the surgical tool unit base 403 so as to slide only with one degree of freedom in linear motion in the x-axis direction, which is the longitudinal direction. Furthermore, the pulley 422 is rotatably supported by the surgical tool unit base 403. The rod 423 as a linear motion transmitting device retreats and advances in the x-axis direction by the drive force transmitted from the side of the drive unit 103. As the rod 423 retreats in the x-axis direction (in other words, advances in the distal direction), the cable 421 connected to the rod 423 is pulled, and can transmit the drive force to the surgical tool 401. The rod 423 is equipped with a rotation inhibiting device so as to inhibit rotation about the longitudinal axis and to operate only with one degree of freedom in linear motion in the longitudinal direction (described below). Furthermore, a spring 424 that applies a force to push the rod 423 in the tip end direction is disposed on the root side of the rod 423 so that the cable 421 is not loosened even in a state where the surgical tool unit 401 is separated from the drive unit 403 to be independent. The rod 423 is inserted into the spring 424, and one end of the spring 424 is secured to the surgical tool unit 403 while the other end is secured to the rod 423. Therefore, when the rod 423 is pushed in the tip end direction by an elastic force of the spring 424, a pre-tension force is applied to the cable 421 folded back by the pulley 422 and coupled to the rod 423. Thus, the cable 421 is not loosened.

It should be understood that linear motion transmitting devices similar to those for the cables 411 and 421 illustrated in FIG. 4 are provided for the cables (not illustrated) that are used in accordance with the degrees of freedom of the surgical tool 401.

Next, the operating principles of the rotational-to-linear motion converting device mounted on the drive unit 103 are described with reference to FIG. 5. The drive unit 103 includes the number of sets of the motor that generates the drive force and the rotational-to-linear motion converting device that converts a rotational motion of the motor into a linear motion, the number corresponding to the number of cables on the side of the surgical tool unit 101. The number of cables on the side of the surgical tool unit 101 varies depending on the degrees of freedom in the configuration of the surgical tool 401 and the like, and accordingly, the number of motors and the number of rotational-to-linear motion converting devices provided on the side of the drive unit 103 also vary. FIG. 5 illustrates the configuration of the motors and the rotational-to-linear motion converting devices for driving the respective cables 411 and 421 of the surgical tool unit 101 illustrated in FIG. 4 in a cross-sectional view of the drive unit 103 taken along a plane including the longitudinal direction. Further, to simplify the drawing, FIG. 5 illustrates the adapter unit 102 integrally with a drive unit base 501 on the side of the drive unit 103. Note that, for convenience sake, the same x- and y-axes as those in FIG. 4 are defined in FIG. 5.

A motor 511 is secured in the drive unit 103 via the drive unit base 501 on the proximal side of the drive unit 103, to drive the rod 413 that pulls the cable 411 on the side of the surgical tool unit 101. In the motor 511, a speed reducer 512 is attached to the output end, and an encoder 513 that measures a rotation angle of the rotation shaft (not illustrated) of the motor 511 is attached to the end surface at the opposite side from the output end. With reduction in size being taken into consideration, an encoder of an incremental type is adopted as the encoder 513, but an encode of an absolute type may be adopted as a matter of course. Further, the motor 511 may also include a brake (not illustrated).

The rotational-to-linear motion converting device that converts rotation of the motor 511 into a linear motion includes: a motor capstan 514 attached to the output shaft of the motor 511 (or the speed reducer 512); a pair of cables 515 and 516 having one ends wound around the motor capstan 514 in opposite directions to each other; a rod 517; and a linear guide 518 that guides the rod 517 so as to slide with respect to the drive unit base 501 only with one degree of freedom in linear motion in the x-axis direction, which is the longitudinal direction. The other end of the cable 515 is rerouted from a circumferential direction of the motor capstan 514 to a negative direction of the x-axis (in the distal direction of the rod 517) via an idler pulley 515A, and is then connected to the distal side of the rod 517. Meanwhile, the other end of the cable 516 is rerouted from the circumferential direction of the motor capstan 514 to a positive direction of the x-axis (in the proximal direction of the rod 517) via an idler pulley 516A, and is then connected to the proximal side of the rod 517. The linear guide 518 is secured to the drive unit base 501 so that the direction in which the rod 517 is guided coincides with the x-axis direction, which is the longitudinal direction. Furthermore, each of the idler pulleys 515A and 515B is rotatably supported by the drive unit base 501.

When the motor 511 rotates forward, one cable 515 is wound around the motor capstan 515. As a result, the rotation is converted into a linear motion in which the rod 517 advances in the negative direction of the x-axis (or the distal side). Furthermore, when the motor 511 rotates backward, the other cable 516 is wound around the motor capstan 514. As a result, the rotation is converted into a linear motion in which the rod 517 retreats in the positive direction of the x-axis (or the proximal side).

When the surgical tool unit 101 is attached to the drive unit 103 via the adapter unit 102, the tip end of the rod 515 on the side of the drive unit 103 comes right into contact with the tip end of the rod 413 on the side of the surgical tool unit 101. Accordingly, when the rod 515 retreats and advances in the x-axis direction by forward rotation and backward rotation of the motor 511, the rod 413 also retreats and advances in the x-axis direction, following the movement of the rod 515. As the rod 515 and the rod 413 retreat together in the x-axis direction (in other words, advances in the distal direction), the cable 411 connected to the rod 413 is pulled, and linearly transmits the drive force to the capstan related to one of the degrees of freedom of the surgical tool 401 serving as an end effector.

Furthermore, a motor 521 is disposed on the proximal side of the drive unit 103, to drive the cable 421 (or the rod 413). A speed reducer 522, an encoder 523, and a brake (not illustrated) are also attached to the motor 521. The rotational-to-linear motion converting device that converts rotation of the motor 521 into a linear motion includes: a motor capstan 524 attached to the output shaft of the motor 511 (or the speed reducer 522); a pair of cables 525 and 526 having one ends wound around the motor capstan 524 in opposite directions to each other; a rod 527; and a linear guide 528 that guides the rod 527 to slide only with one degree of freedom in linear motion in the longitudinal direction with respect to the drive unit base 501. The other end of the cable 525 is rerouted from a circumferential direction of the motor capstan 524 to the negative direction of the x-axis (in the distal direction of the rod 527) via an idler pulley 525A, and is then connected to the distal side of the rod 517. Meanwhile, the other end of the cable 526 is rerouted from the circumferential direction of the motor capstan 524 to the positive direction of the x-axis (in the proximal direction of the rod 527) via an idler pulley 526A, and is then connected to the proximal side of the rod 527. The linear guide 528 is secured to the drive unit base 501 so that the direction in which the rod 527 is guided coincides with the x-axis direction, which is the longitudinal direction. Furthermore, each of the idler pulleys 525A and 525B is rotatably supported by the drive unit base 501.

When the motor 521 rotates forward, one cable 525 is wound around the motor capstan 525. As a result, the rotation is converted into a linear motion in which the rod 527 advances in the negative direction of the x-axis (or the distal side). Furthermore, when the motor 521 rotates backward, the other cable 516 is wound around the motor capstan 524. As a result, the rotation is converted into a linear motion in which the rod 527 advances in the positive direction of the x-axis (or the proximal side).

When the surgical tool unit 101 is attached to the drive unit 103 via the adapter unit 102, the tip end of the rod 525 comes right into contact with the tip end of the rod 423 on the side of the surgical tool unit 101. Accordingly, when the motor 521 rotates forward and the rod 525 retreats in the x-axis direction (in other words, advances in the distal direction), the cable 421 is pulled via the rod 423, and linearly transmits the drive force to the capstan related to one of the degrees of freedom of the surgical tool 401.

It should be understood that the motors (not illustrated) corresponding to the respective cables that are used in accordance with the degrees of freedom of the surgical tool 401 are also equipped with rotational-to-linear motion converting devices similar to those for the motors 511 and 521 described above, and linearly transmit the drive force to the side of the surgical tool unit 101.

Note that, in the art, ball screws, racks, and pinion mechanisms are also known as rotational-to-linear motion converting devices that convert a rotational motion of a motor into a linear motion. In a case where a ball screw, a rack, or a pinion mechanism is used, a problem of backlash and a problem of backdrivability occur, as described in the above section B. When backdrivability is low, it is difficult to detect an external force acting on an end effector such as a surgical tool on the root side, for example. Also, when a ball screw, a rack, or a pinion mechanism is used, the structure becomes more complicated, and it becomes more difficult to reduce the size and the weight of the entire surgical tool device.

On the other hand, the rotational-to-linear motion converting device using cable driving of rods as illustrated in FIG. 5 can operate without any backlash but with a high backdrivability, and is also suitable for a surgical robot that requires precise force control, for example. Furthermore, as the rotational-to-linear motion converting device is disposed on the side of the drive unit 103, the surgical tool unit 101 as a replacement part can be made smaller in size and diameter, the surgical tool stand accommodating a plurality of surgical tool units can be made smaller in size and be disposed near the arm device, and the motion range of the arm device for changing surgical tools can be smaller.

Note that the terms used in the present specification are now briefly explained. A “capstan” and an “idler pulley” are both pulleys. A pulley that is used for cable layout adjustment and application of tension to a cable is referred to as an “idler pulley” in the present specification. Furthermore, a pulley that is used for application of power to a cable or conversely for conversion of a force from a cable into an axial force is called a “capstan” in the present specification, and an input capstan and an output capstan are both pulleys that are used in this use application.

### D. Detailed Configuration of a Surgical Tool Unit

In this section D, a detailed configuration of a surgical tool unit capable of operating a surgical tool at the distal end with a linear motion force supplied from the drive unit side is described. Since the surgical tool unit does not include a rotational-to-linear motion converting device, it is easy to reduce the size and the diameter thereof. Accordingly, more surgical tool units can be mounted on the surgical tool stand, to improve the degree of integration and the like. Further, when a robot automatically changes surgical tool units, the robot can perform accurate attachment even if the robot roughly inserts the root of the surgical tool unit into the tip end of the drive unit, as long as the surgical tool unit is small in size and diameter.

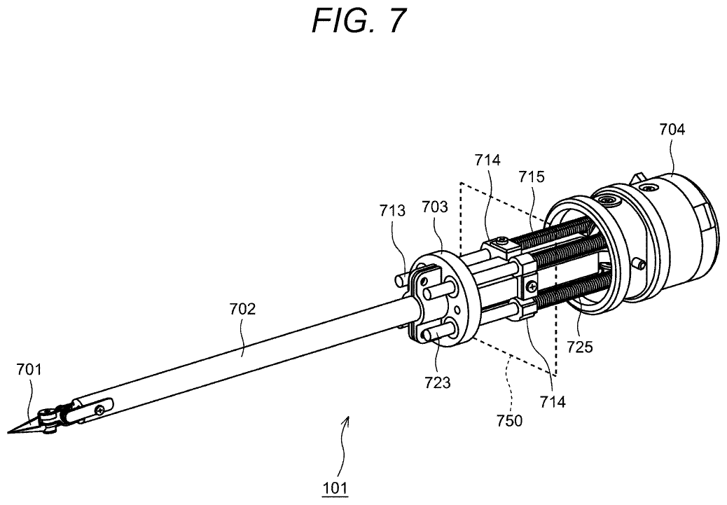

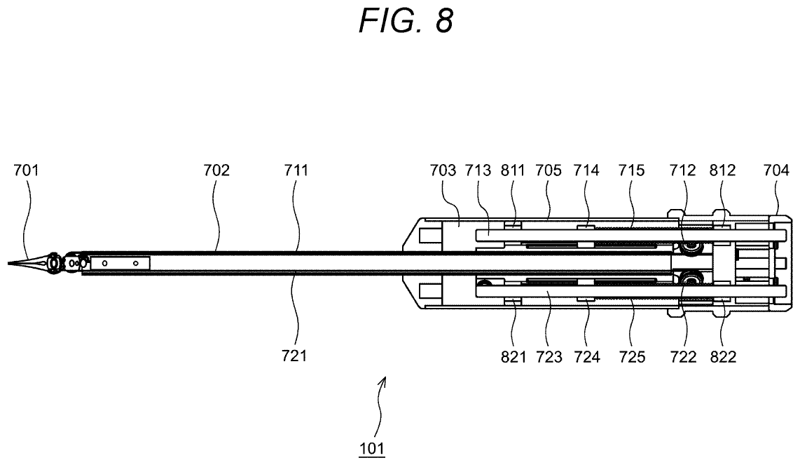

FIG. 7 is a perspective view illustrating a specific configuration of the surgical tool unit 101. Further, FIG. 8 illustrates an internal configuration of the surgical tool unit 101 illustrated in FIG. 7, with a cross-sectional view taken along a plane including the longitudinal direction. The basic configuration and operation of the surgical tool unit 101 illustrated in FIGS. 7 and 8 are as described in the above section C with reference to FIG. 4.

The surgical tool unit 101 includes a surgical tool 701, a hollow shaft 702 that supports the surgical tool 701 at the tip end (or the distal end), and an inner base 703 that supports the shaft 702. The inner base 703 is secured to a joining portion 704 for being joined to the adapter unit 102 on the root side (or the proximal side), and the periphery thereof is covered with a cylindrical case 705. Note that, in FIG. 7, the case 705 is not illustrated, to clarify the internal structure.

The surgical tool 701 is not limited to any particular kind, but may be forceps including a pair of jaws disclosed in Patent Document 2 or Patent Document 3, for example. Each of the jaws is rotatably supported about the opening/closing axis (or the yaw axis) near the tip end of the shaft, and a torsion spring (or some other elastic member) that applies beforehand an opening force acting in a direction of opening (or in a direction where the jaws are separated from each other) about the opening/closing axis between the jaws is incorporated into the opening/closing axis (or the yaw axis). The forceps including the pair of jaws is then supported so as to be rotatable about a pitch axis (wrist) orthogonal to each of the opening/closing axis and the longitudinal axis of the shaft.

One end of a cable 711 on the tip end side (or the distal side) is connected to a capstan (not illustrated in FIG. 7) related to one of the degrees of freedom of the surgical tool 701 serving as an end effector. Furthermore, the other end of the cable 711 extending to the root side (or the proximal side) is inserted into the shaft 702, is then drawn into the inner base 703, is wound around a pulley 712 on the root side of the surgical tool unit 101 (or the shaft 702), is folded back in the direction of the distal end (or the tip end), and is then connected to a rod 713 via a cable coupling portion (described below) (or the vicinity of the end is secured).

A cable 721 is similar to the cable 711. One end of the cable 721 on the tip end side (or the distal side) is connected to a capstan (not illustrated) related to one of the degrees of freedom of the surgical tool 701, and the other end of the cable 721 extending to the root side (or the proximal side) is inserted into the shaft 702, is then drawn into the inner base 703, is wound around a pulley 722 on the root side of the surgical tool unit 101 (or the shaft 702), is folded back, and is then connected to a rod 723 via a cable coupling portion (described later) (or the vicinity of the end is secured).

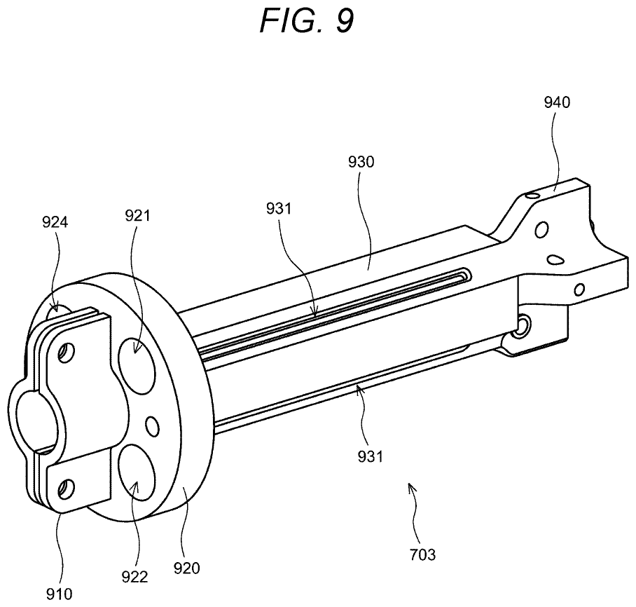

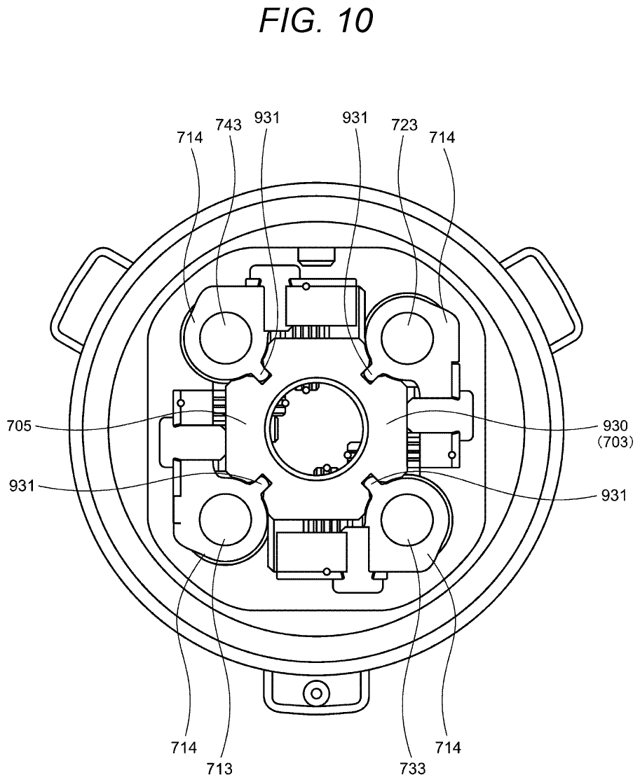

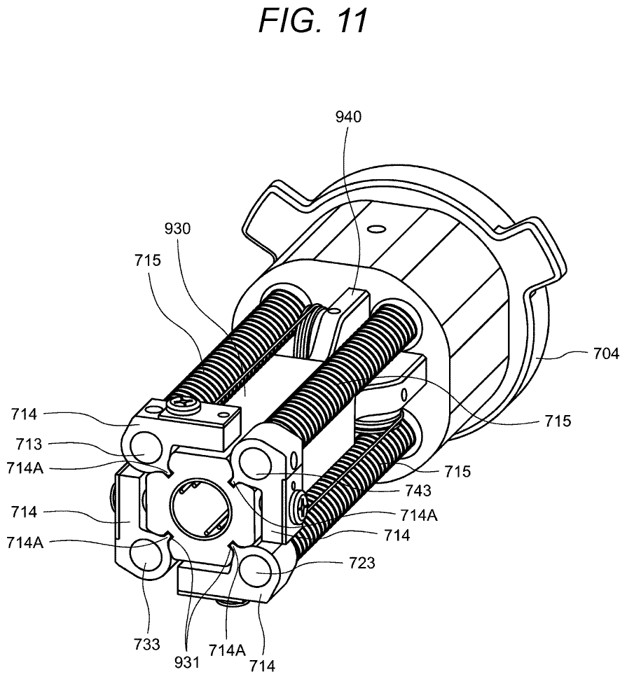

Further, FIG. 9 illustrates a perspective view of only the inner base 703 extracted from FIG. 7. Further, FIG. 10 illustrates a cross-sectional view of the surgical tool unit 101 taken along a plane orthogonal to the longitudinal direction, as indicated by reference numeral 750 in FIG. 7. Furthermore, FIG. 11 illustrates a perspective view of the base side of the surgical tool unit 101 taken along the plane 750. The plane 750 partitions the surgical tool unit 101 exactly at the position where a rotation inhibiting device 714 (described later) is disposed. Further, FIG. 12 is an exploded view of the surgical tool unit 101 exploded in the longitudinal direction.

As illustrated in FIG. 9, the inner base 703 includes, on the tip end side, a shaft connecting portion 910 to which the shaft 702 is mounted, and a disk-like rod support portion 920 in which four rod insertion holes 921 to 924 through which four rods including the rods 713 and 723 are inserted in the longitudinal direction are drilled. The shaft connecting portion 910 includes two symmetrical plate members each having a central attachment portion that forms a semi-cylindrical space. As these plate members are combined, a cylindrical space for attaching the shaft 702 is formed in the center. After the root portion of the shaft 702 is inserted into the cylindrical space, the two plate members are screwed, so that the shaft connecting portion 910 can sandwich the root portion of the shaft 702. Furthermore, in the inner base 703, a pulley support portion 940 that rotatably supports four idler pulleys for folding back four cables including the pulleys 712 and 722 toward the distal end side is disposed on the root side, and the rod support portion 920 and the pulley support portion 940 are connected via a frame 930. In the frame 930, four guide grooves including a guide groove 931 are formed in the longitudinal direction of the four rods including the rods 713 and 723. These guide grooves each has a length that covers a longitudinal movable range of the corresponding rod. Note that, for ease of explanation, the inner base 703 is divided into four portions 910 to 940, and each portion is named. However, it is assumed that the inner base 703 is manufactured as one integrated component. The inner base 703 may of course be formed with a combination of a plurality of components.

As can be seen from the cross-sectional view illustrated in FIG. 8, the rod 713 is supported at the two positions of the rod insertion hole 921 of the rod support portion 920 of the inner base 703 and the joining portion 704 on the root side via bearings 811 and 812, respectively. Each of these bearings 811 and 812 is formed with a plain bearing, and guides a linear motion of the rod 713 in the longitudinal direction. Note that the inner base 703 may be designed to be supported by plain bearings at three or more positions. Likewise, the rod 723 is supported at the two positions of the rod insertion hole 921 of the rod support portion 920 of the inner base 703 and the joining portion 704 on the root side via bearings 821 and 822, respectively.

As described in the above section C with reference to FIG. 5, the rod 713 linearly moves (in other words, retreats and advances) in the longitudinal direction with the drive force transmitted from the side of the drive unit 103. As the rod 713 advances in the distal direction, the cable 711 connected to the rod 713 is pulled, and transmits the drive force to the capstan related to one of the degrees of freedom of the surgical tool 701 serving as an end effector. Thus, the rod 713 serves as a linear motion transmitting device. However, the specific configuration of the drive unit 103 will be described in the next section E.

As can be seen from FIGS. 7 and 8, the rotation inhibiting device 714 is attached to the rod 713 at an intermediate position between the two positions at which the rod 713 is supported by the bearings 811 and 812. FIG. 13 illustrates a perspective view of only one rod 713 extracted from the surgical tool unit 101 illustrated in FIG. 7. The specific shape and structure of the rotation inhibiting device 714 will be more apparent from FIG. 13. A principal role of the rotation inhibiting device 714 is to inhibit rotation about the longitudinal axis when the rod 713 is guided by the bearings 811 and 812 and linearly moves. This is because, if the rod 713 rotates about the longitudinal axis, the cable 711 having the other end secured to the rod 713 is wound around the rod 713, and cannot accurately drive the surgical tool 701 in accordance with the amount of linear motion of the rod 713 in the longitudinal direction.

As illustrated in FIG. 13, the rotation inhibiting device 714 has a protrusion 714A protruding inward. Further, as already described with reference to FIG. 9, in the frame 930, the guide groove 931 is formed in the longitudinal direction of the rod 713. In a state where the rod 713 to which the rotation inhibiting device 714 is attached is incorporated into the surgical tool unit 101, the protrusion 714A is fitted right in the guide groove 931. The guide groove 931 has a length that covers the movable range of the rod 713 in the longitudinal direction. Accordingly, the rod 713 linearly moves in the longitudinal direction while the protrusion 714A is guided by the guide groove 931, so that rotation about the longitudinal axis of the rod 713 can be inhibited.

Note that the protrusion-recess relationship may be reversed from that described above, a linear protrusion having a length covering the movable range of the rod 713 in the longitudinal direction may be provided in the frame 930, and a groove for sliding the linear protrusion may be formed on the side of the rotation inhibiting device 714, to guide the linear protrusion along the groove on the side of the rotation inhibiting device 714 when the rod 713 linearly moves. In this manner, the rotation inhibiting device 714 can also inhibit rotation of the rod 713 about the longitudinal axis.

Furthermore, the rotation inhibiting device 714 also serves as a cable coupling portion that connects (or secures) the other end of the cable 711 wound around the pulley 712 and folded back in the direction of the distal end (or the tip end) (note that, in FIG. 13, the cable 711 is not illustrated, to avoid confusion on the drawing). Note that the other end of the cable 711 may be secured to the rod 713 by a cable coupling portion formed as a different component from the rotation inhibiting device 714. Further, in the example illustrated in FIGS. 7 to 13, the rod 713 and the rotation inhibiting device 714 are formed as separate components, but the rod 713 and the rotation inhibiting device 714 may be formed as an integrated component.

Furthermore, a spring 715 that applies a force to push the rod 713 in the tip end direction is disposed on the root side of the rod 713 so that the cable 711 is not loosened even in a state where the surgical tool unit 101 is separated from a drive unit 703 to be independent. Specifically, as can be seen from FIG. 12, the root side of the rod 713 is inserted into the spring 715, and, in a state where the surgical tool unit 101 is assembled, the root side of the spring 715 is in contact with the tip end surface of the joining portion 704, while the tip end side of the spring 715 is in contact with the rotation inhibiting device 714. Accordingly, the rotation inhibiting device 714 also serves as a surface to be in contact with the spring 715. In a state where the surgical tool unit 101 is separated from the drive unit 703, the rod 713 is then pushed in the tip end direction by the elastic force of the spring 715, and a pre-tension force is applied to the cable 711 folded back by the pulley 712, so that the cable is not loosened.

Although the motion of the rod 713 and the rotation inhibiting device 714 related to the rod 713 have been mainly described above, it should be understood that each of the motion, the mechanism of the rotation inhibiting device, and the pre-tension applying spring for the cable is also similar for the other three rods and cables including the rod 723 and the cable 721.

### E. Detailed Configuration of a Drive Unit

In this section E, a detailed configuration of a drive unit is described. A surgical tool unit is attached to a drive unit via an adapter unit, and the drive unit supplies linear motion forces to the surgical tool unit. The drive unit is equipped with the number of motors corresponding to the number of linear motion forces (four in the present embodiment) required on the surgical tool unit side, and a rotational-to-linear motion converting device that converts a rotative force of the motor into a linear motion force.

In the present disclosure, with the use of a rotational-to-linear motion converting device that operates without any backlash but with a high backdrivability using cable driving, a surgical tool device is easily applied to a surgical robot that performs delicate work. When backdrivability is high, an external force acting on the surgical tool unit (or the surgical tool at the distal end thereof) can be measured on the basis of the current value of the motor on the drive unit side.

Since the rotational-to-linear motion converting device using cable driving according to the present disclosure has a simple structure and achieves reduction in size and weight, a drive unit equipped with this rotational-to-linear motion converting device and the entire arm device are made smaller in size and weight, and are easily applied to a wide variety of operating rooms. As the arm device is made smaller in size, the motion range of the arm device at a time of automatic surgical tool replacement becomes smaller. Thus, the risk of contact with the environment can be lowered, and the necessary work space can be made smaller. Further, as the rotational-to-linear motion converting device is mounted on the drive unit side, instead of the surgical tool unit side, the cost of each surgical tool unit as a replacement part is prevented from becoming higher, and the surgical tool units can be easily made smaller in size and diameter.

As described above in the section B with reference to FIGS. 1 and 2, the drive unit 103 includes the number of sets of the motor that generates the drive force and the rotational-to-linear motion converting device that converts a rotational motion of the motor into a linear motion, the number corresponding to the number of cables on the side of the surgical tool unit 101. In the present embodiment, forceps including a pair of jaws is assumed as an end effector to be attached to the distal end of the surgical tool unit 101, and the surgical tool unit 101 in this case uses a total of four cables including two cables for pulling each jaw around the opening/closing axis and two cables for rotating the forceps forward and backward about the pitch axis (wrist) (as described above). Therefore, as illustrated in FIGS. 1 and 2, only four sets of a motor and a rotational-to-linear motion converting device are mounted on the drive unit 101.

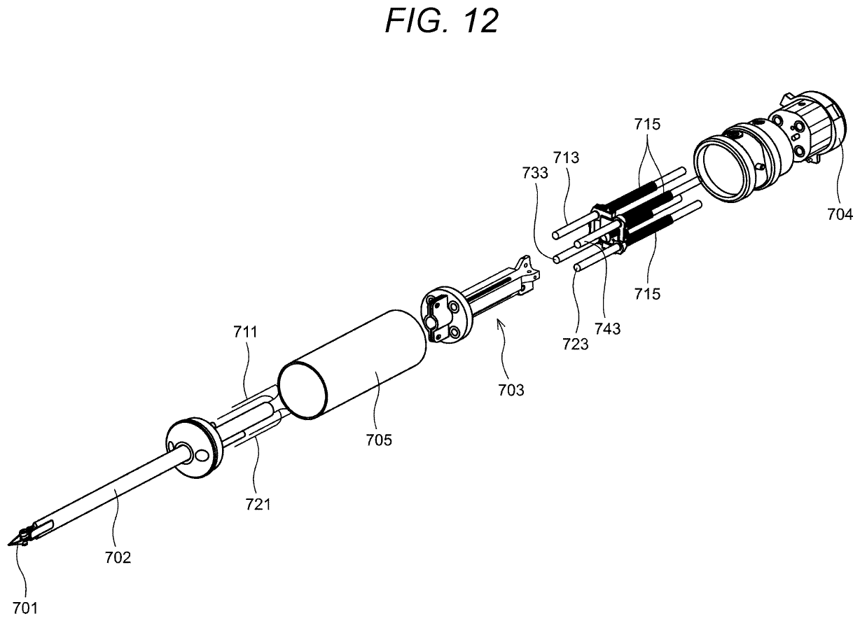

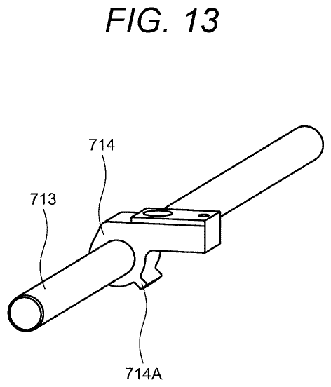

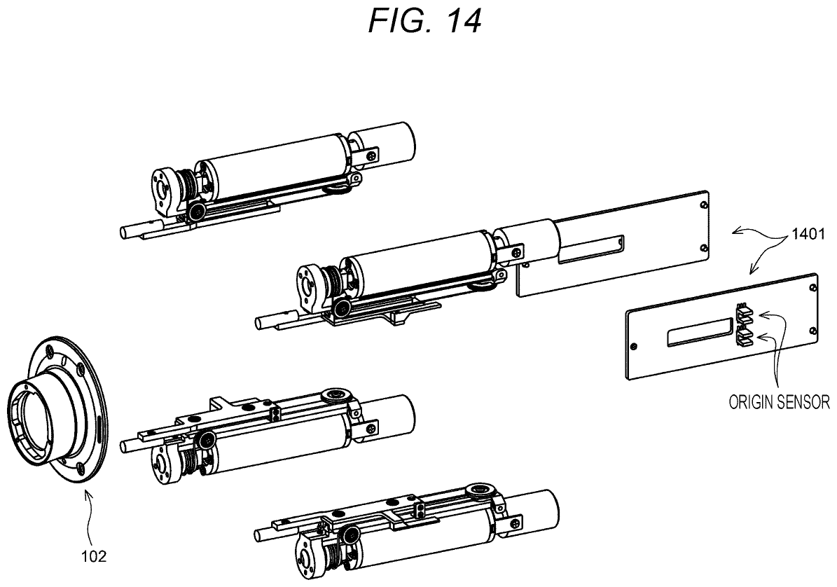

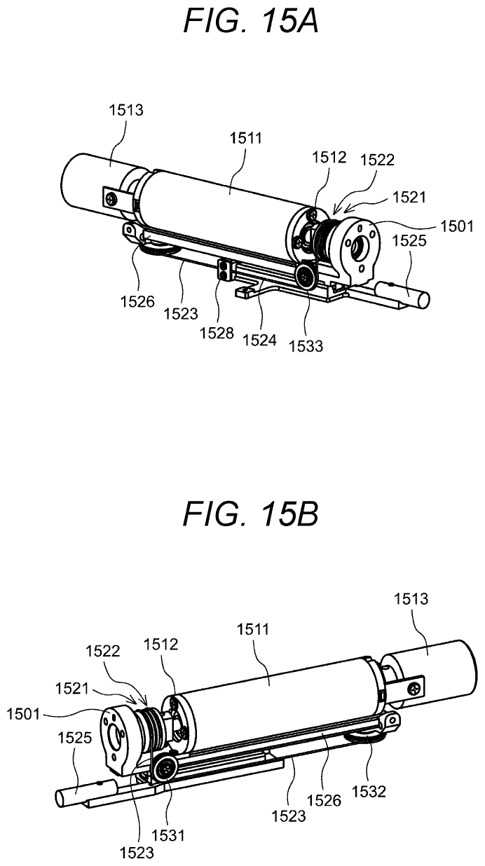

FIG. 14 illustrates an image in which four sets of a motor and a rotational-to-linear motion converting device on the side of the drive unit 103 are disposed for the adapter unit 102. Further, FIGS. 15A and 15B illustrate states in which one side surface and the opposite side surface orthogonal to the longitudinal direction of one set of a motor and a rotational-linear motion device among the four sets are viewed. In the description below, the structure and operation of one set of a motor and a rotational-linear motion device will be described with reference to FIGS. 15A and 15B. However, it should be understood that the same applies to the other three sets.

Referring to FIG. 15, a motor 1511 is secured in the drive unit 103 via a drive unit base 1501 on the proximal side of the drive unit 103, to drive a rod (not illustrated) that pulls a cable on the side of the surgical tool unit 101. In the motor 1511, a speed reducer 1512 is attached to the output end, and an encoder 1513 that measures a rotation angle of the rotation shaft (not illustrated) of the motor 1511 is attached to the end surface at the opposite side from the output end. With size reduction being taken into consideration, an encoder of an incremental type is adopted for the encoder 1513. Therefore, as illustrated in FIG. 14, a printed wiring board 1401 including an origin sensor for detecting the origin position of the rotational-to-linear motion converting device that is driven by a motor 1501 is provided for each motor. Further, the motor 1511 may also include a brake (not illustrated).

The rotational-to-linear motion converting device that converts rotation of the motor 1511 into a linear motion includes: a capstan assembly including a pair of front and rear motor capstans 1521 and 1522 coaxially attached to the output shaft of the motor 1511 (or the speed reducer 1512); cables 1523 and 1524 wound around the motor capstans 1521 and 1522, respectively; a slide base 1526 to which a rod 1525 is integrally attached (hereinafter, the rod 1525 and the slide base 1526 will be described as an integrated component); a linear guide that guides the slide base 1526 to slide with respect to the drive unit base 1501 only with one degree of freedom in linear motion in the x-axis direction, which is the longitudinal direction; and three idler pulleys 1531 to 1533 for changing paths of the cables 1523 and 1524. Note that, although it is difficult to visually recognize the linear guide in FIG. 15, the linear guide corresponds to the linear guides 518 and 528 in FIG. 5, and is disposed between the slide base 1526 integrated with the rod 1525 and the drive unit base 1501. Each of the idler pulleys 1531 to 1533 is rotatably supported by the drive unit base 1501. The idler pulley 1531 and the idler pulley 1532 have the same horizontal rotation axis orthogonal to the rotation axis of the motor 1511, and the idler pulley 1532 has a vertical rotation axis orthogonal to the rotation axis of the motor 1511.

The respective end portions of the cables 1523 and 1524 are secured to a side surface of the slide base 1526 by a cable securing portion 1528 from directions opposite to each other in the longitudinal direction. First, the layout of the cable 1523 is described. One end of the cable 1523 is secured to the front motor capstan 1521, and is wound around the motor capstan 1521 in the direction in which the cable 1523 is wound by forward rotation of the motor 1511. When the cable 1523 is separated from the circumference of the motor capstan 1521, the cable 1523 is rerouted rearward in the longitudinal direction of the rod 1525 by the idler pulley 1531, and is further rerouted forward in the longitudinal direction by the idler pulley 1532 near the rear end of the motor 1511. The other end of the cable 1523 is then secured to the side surface of the slide base 1526 by the cable securing portion 1528.

Next, the layout of the cable 1524 is described. One end of the cable 1524 is secured to the rear motor capstan 1522, and is wound around the motor capstan 1522 in the direction in which the cable 1524 is wound by backward rotation of the motor 1511. When the cable 1524 is separated from the circumference of the motor capstan 1522, the cable is then rerouted rearward in the longitudinal direction of the rod 1525 by the idler pulley 1533, and the other end of the cable 1524 is secured to the side surface of the slide base 1526 by the cable securing portion 1528.

When the motor 1511 rotates forward, one cable 1523 is wound around the motor capstan 1521, so that the linear guide causes the slide base 1526 to slide in the positive direction in the x-axis direction with respect to the drive unit base 1501. As a result, the forward rotation is converted into a linear motion in which the rod 1525 retreats toward the proximal side. Further, when the motor 1511 rotates backward, the other cable 1524 is wound around the motor capstan 1522, so that the linear guide causes the slide base 1526 to slide in the negative direction in the x-axis direction with respect to the drive unit base 1501. As a result, the backward rotation is converted into a linear motion in which the rod 1525 advances toward the distal side.

It is assumed that any set of the motor and the rotational-to-linear motion converting device illustrated in FIG. 14 has a configuration and operation as described above with reference to FIGS. 15A and 15B. As can be also seen from FIG. 14, each set among the four motors and the four rotational-to-linear motion converting devices is connected to the adapter unit 102, and the printed wiring board 1401 having the origin sensor mounted thereon is incorporated. The assembling method by which the respective motors and the respective rotational-to-linear motion converting devices are individually assembled and are then attached to the adapter unit 102 is easy, and also excels in maintainability.

When the surgical tool unit 101 is attached to the drive unit 103 via the adapter unit 102, the tip end of each rod on the side of the drive unit 103 comes right into contact with the tip end of each corresponding rod on the side of the surgical tool unit 101. Accordingly, when a motor rotates backward, the rod advances in the negative direction of the x-axis, which is the direction of the distal side. As a result, the corresponding rod on the side of the surgical tool unit 101 is pushed toward the distal side, and the cable connected the rod is pulled. Thus, the drive force can be linearly transmitted to the surgical tool at the distal end.

The rotational-to-linear motion converting device using cable driving according to the present disclosure can operate without any backlash but with a high backdrivability compared with a ball screw, a rack, and a pinion mechanism, and is also suitable for a surgical robot that requires precise force control, for example. As illustrated in FIGS. 14, 15A, and 15B, as the rotational-to-linear motion converting device is disposed on the side of the drive unit 103, the surgical tool unit 101 as a replacement part can be made smaller in size and diameter, the surgical tool stand accommodating a plurality of surgical tool units can be made smaller in size and be disposed near the arm device, and the motion range of the arm device for changing surgical tools can be smaller.

### F. Downsizing of a Rotational-to-Linear Motion Converting Device

Referring to FIGS. 15A and 15B, for example, when the cable 1523 and the cable 1524 are loosened, these cables are derailed from the idler pulley 1531 and the idler pulley 1533, respectively, or cause backlash. As a result, the linear drive force cannot be supplied from the drive unit 103 to the surgical tool unit 101, or the surgical tool at the distal end cannot be accurately operated. For this reason, a structure for applying a pre-tension force to the cables 1523 and 1524 is necessary.

One of the common means is to apply a pre-tension force to a cable by connecting tension coil springs in series (see Patent Document 2, for example). However, in a case where this means is used, a space equivalent to the length of the tension coil springs is required in the path of the cable. Referring to FIGS. 15A and 15B, to connect the tension coil springs in series to the cable 1523, at least one of the distances between the idler pulley 1531 and the idler pulley 1532, and between the idler pulley 1532 and the cable securing portion 1528 needs to be widened by the amount equivalent to the length of the tension coil springs. Further, to connect the tension coil springs in series to the cable 1524, it is necessary to widen the distance between the cable securing portion 1528 and the idler pulley 1533 by the amount equivalent to the length of the tension coil springs. Because of this, the rotational-to-linear motion converting device becomes longer. Furthermore, the outer diameter of the coil springs is larger than the diameters of the cables 1523 and 1524, and therefore, the width of the rotational-to-linear motion converting device also tends to become greater.

Therefore, in the present disclosure, the motor capstan attached to the output shaft of the motor 1511 is divided into the two components of the pair of front and rear motor capstans 1521 and 1522 as described above, and a torsion spring is sandwiched between the motor capstan 1521 and the motor capstan 1522. With such a configuration, the rotative force in the direction of winding the cables 1523 and 1524 can be applied to the motor capstan 1521 and the motor capstan 1522 with the restoring force of the torsion spring.

FIG. 16 illustrates, in an enlarged view, the motor capstans 1521 and 1522 attached to the output shaft of the motor 1511 of the rotational-to-linear motion converting device illustrated in FIG. 15B, and illustrates a state in which the motor capstan 1521 and the motor capstan 1522 are disassembled, and further a state in which a torsion spring 1601 is attached to the inside of the motor capstan 1521 (or 1522).

As can be seen from the exploded view in FIG. 16, the motor capstan 1521 and the motor capstan 1522 each include a space for attachment of the torsion spring 1601 therein. The structure in each space is symmetrical between the motor capstan 1521 and the motor capstan 1522. Each space has hollow shaft portions 1611 and 1612 attached to the output shaft of the motor 1511 at the center, and protrusions 1621 and 1622 for locking the end portion of the torsion spring 1601 are formed at one portion of the outer wall portion.

The motor capstan 1521 and the motor capstan 1522 are assembled with the torsion spring 1601 interposed in between in such a manner that the coil portion of the torsion spring 1601 is inserted into the respective shaft portions 1611 and 1612 of the motor capstan 1521 and the motor capstan 1522, and the respective end portions of the torsion spring 1601 are caught by the protrusions 1612 and 1622 of the motor capstans 1521 and 1522. In such a manner, a restoring force to rotate the motor capstan 1521 and the motor capstan 1522 in opposite directions acts on the torsion spring 1601. That is, a rotative force in the direction of winding the cable 1523 constantly acts on one motor capstan 1521, and a pre-tension force can be applied to the cable 1523. Also, a rotative force in the direction of winding the cable 1524 constantly acts on one motor capstan 1522, and a pre-tension force can be applied to the cable 1524. Note that an elastic member other than the torsion spring 1601 may be incorporated into the capstan assembly, to apply rotative forces in directions opposite to each other to the motor capstan 1521 and the motor capstan 1522.

With a pre-tension applying mechanism as illustrated in FIG. 16, the rotational-to-linear motion converting devices do not become longer by the amount equivalent to the length of the tension coil springs, and the width of the rotational-to-linear motion converting devices does not become greater by the amount equivalent to the outer diameter of the tension coil springs, compared with the method by which tension coil springs are connected in series to a cable. Thus, according to the present disclosure, it is possible to avoid an increase in size of the rotational-to-linear motion converting devices due to application of a pre-tension force to a cable.

### G. Attaching/Detaching Structure, Attaching Procedures, and Detaching Procedures for a Surgical Tool Unit

As described in the above sections E and F, the rotational-to-linear motion converting devices disposed in the drive unit 103 can be downsized, and the drive unit 103 and the entire arm device can be made significantly smaller in size and weight. Further, the motion range of the arm device at a time of replacing the surgical tool unit 101 becomes smaller. Thus, the risk of destroying the other side at a time of contact with the environment or at a time of an out-of-control operation is lowered, and safety can be enhanced. In this section G, an attaching/detaching structure for the surgical tool unit 101, and attaching procedures and detaching procedures for the drive unit 103 are described.

### G-1. Attaching/Detaching Structure for a Surgical Tool Unit

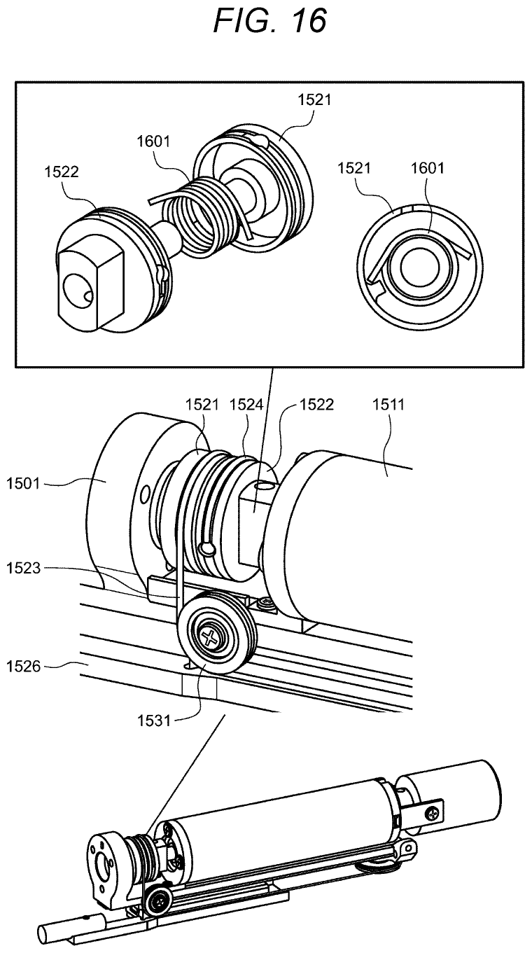

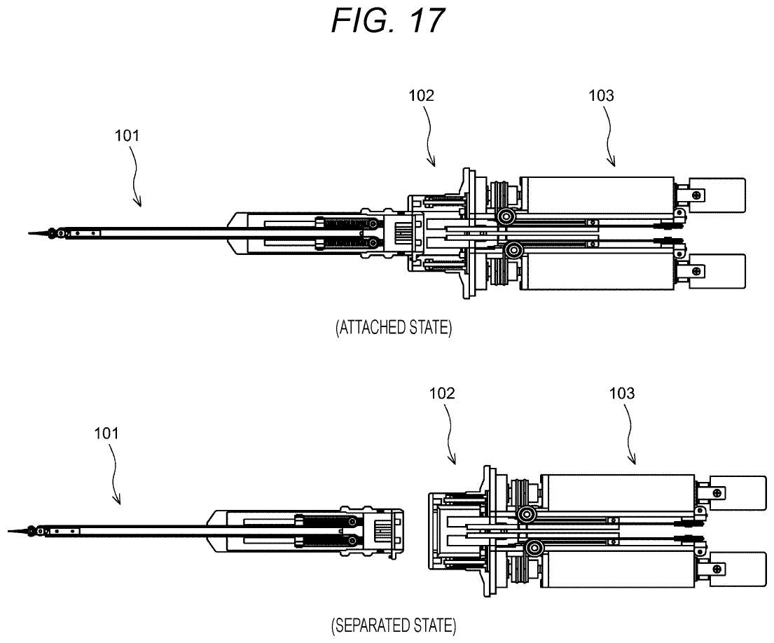

FIG. 17 illustrates cross-sectional views of the respective surgical tool devices 100 in a state where the surgical tool unit 101 is attached to the drive unit 103 and a state where the surgical tool unit 101 is separated from the drive unit 103. As described in the above section B with reference to the perspective views illustrated in FIGS. 1 and 2, the surgical tool unit 101 is attached to the drive unit 103 via the adapter unit 102 in a replaceable manner.

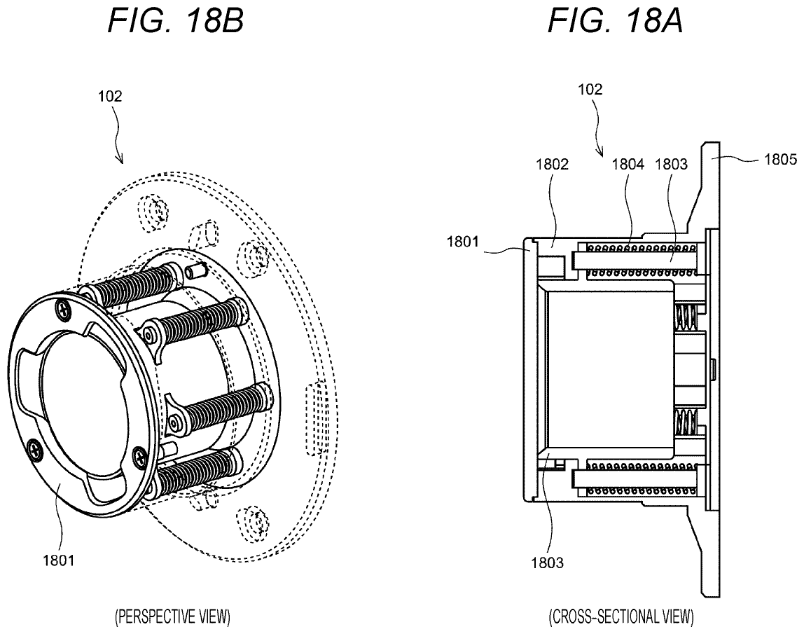

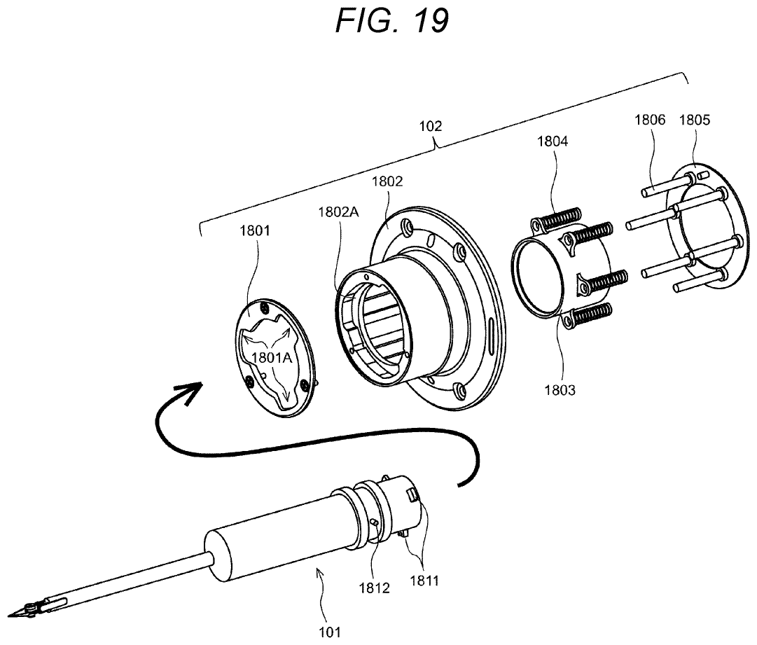

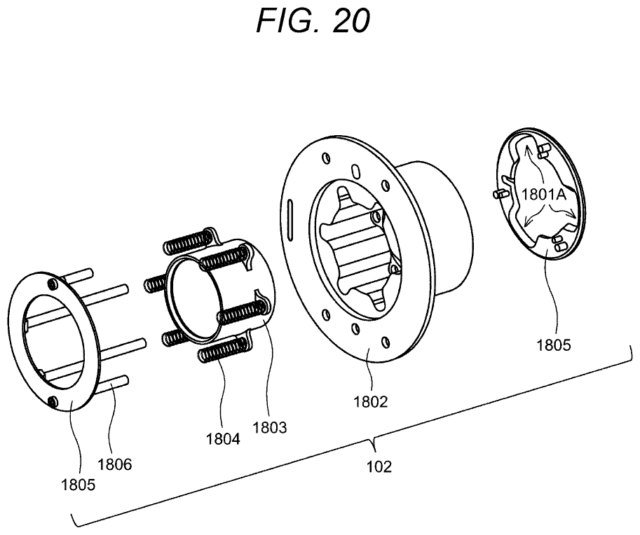

FIGS. 18A and 18B illustrate a cross-sectional view of the adapter unit 102 taken along a plane including the longitudinal axis, and a perspective view of the adapter unit 102 as viewed from the distal side (note that, in FIG. 18B, the internal configuration is partially made transparent, and the contours are indicated by dotted lines, to clarify the internal configuration). Further, FIG. 19 illustrates an exploded view of the adapter unit 102 exploded in the longitudinal direction (note that an image of attachment of the surgical tool unit 101 is also illustrated). For reference, FIG. 20 illustrates an exploded view of the adapter unit 102 as viewed from the proximal side, which is the opposite side in FIG. 19.

As illustrated in FIG. 19, the adapter unit 102 includes, in order from the distal side, a front plate 1801, an adapter base 1802, a pressing device 1803, a plurality of springs 1804 that pushes out the pressing device 1803 to the distal side, and a spring securing plate 1805 serving as a contact surface for the other end on the proximal side of each spring 1804. The spring securing plate 1805 includes a plurality of guide shafts 1806 through which the respective springs 1804 are inserted to determine positions in the extending and contracting directions.

Furthermore, as illustrated in FIG. 19, the surgical tool unit 101 includes a plurality of claws 1811 and a rotation positioning pin 1812 on the outer periphery on the root side. The front plate 1801 has a plurality of recesses 1801A for allowing the respective claws 1811 on the side of the surgical tool unit 101 to pass therethrough, and a positioning groove 1801B that is engaged with the rotation positioning pin 1812 on the side of the surgical tool unit 101 to determine attachment positions of the surgical tool unit 101 and the adapter unit 102. The pressing device 1803 is biased in the distal direction by the plurality of springs 1804 arranged in the circumferential direction. When the respective claws 1811 pass through the recesses 1801A, and the root portion of the surgical tool unit 101 is inserted into the adapter base 1802, the pressing device 1803 presses the respective claws 1811 of the surgical tool unit 101 against the back surface of the front plate 1801. The adapter base 1802 has a counterbore surface 1802A that comes into contact with the respective claws 1811 on the side of the surgical tool unit 101.

FIG. 21 is an enlarged view of the vicinity of the root of the surgical tool unit 101 attached to the adapter unit 102. Further, the upper portion, the middle portion, and the lower portion of FIG. 22 illustrate front views of the respective states before, in the middle of, and at the position of attachment of the surgical tool unit 101 to the adapter unit 102 as viewed from the distal end side.

As illustrated in the upper portion of FIG. 22, when alignment is performed so that the longitudinal axes of the surgical tool unit 101 and the adapter unit 102 coincide with each other, and the respective claws 1811 at the root of the surgical tool unit 101 are aligned with the recesses 1801A of the front plate 1801 at rotation positions, the surgical tool unit 101 can be inserted into the central opening of the front plate 1801.

Next, as illustrated in the middle portion of FIG. 22, when the surgical tool unit 101 is rotated clockwise with respect to the adapter unit 102 toward the attachment position, the clearance between the outer diameter of the claws 1811 and the counterbore surface 1802A of the adapter base 1802 decreases, and the surgical tool unit 101 and the adapter unit 102 are coaxially secured as illustrated in the lower portion of FIG. 22.

### G-2. Attaching Procedures for a Surgical Tool Unit

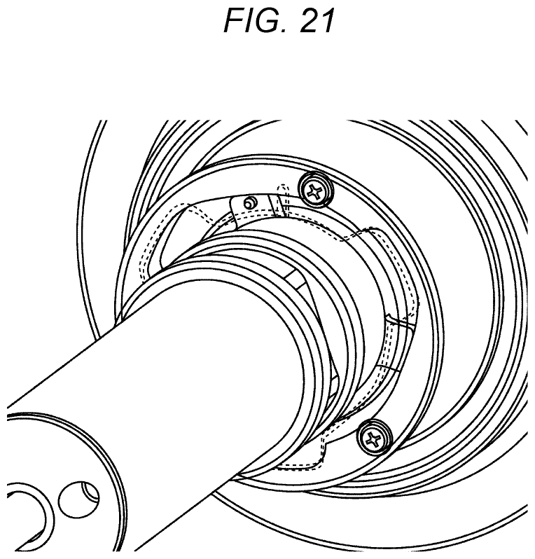

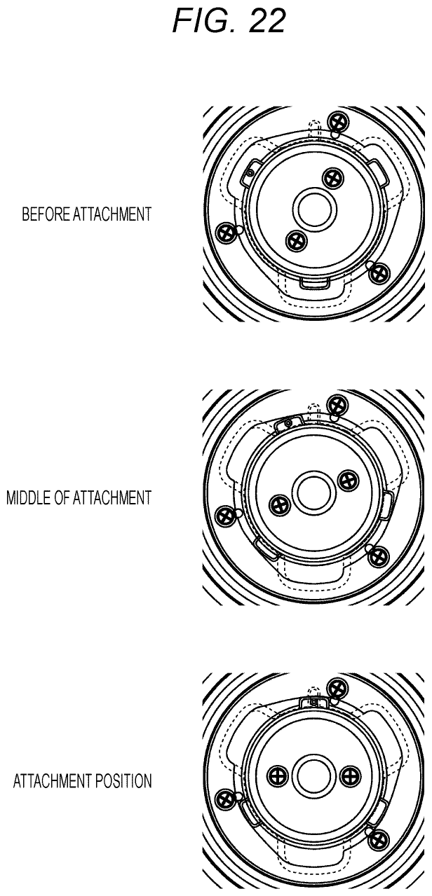

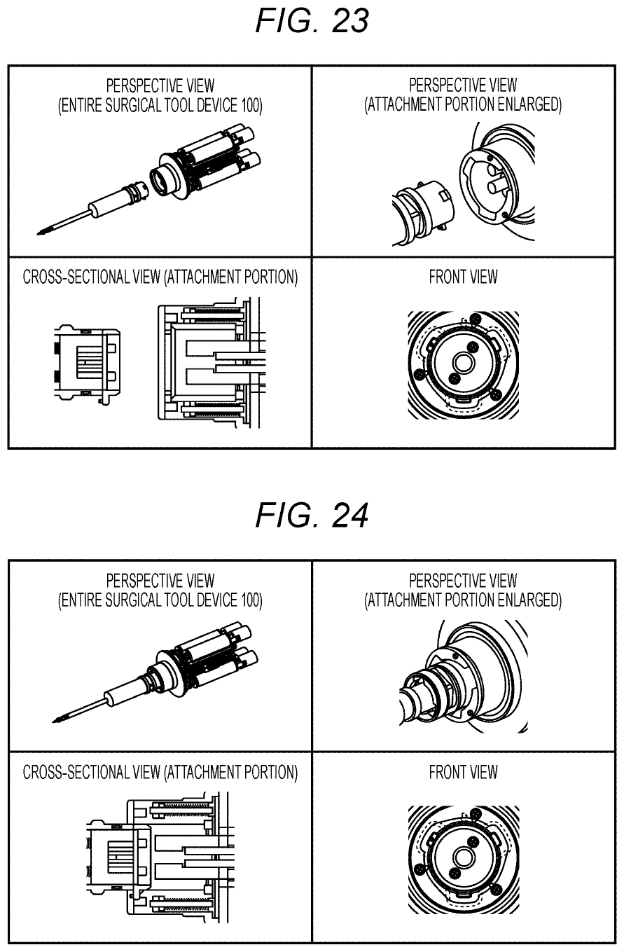

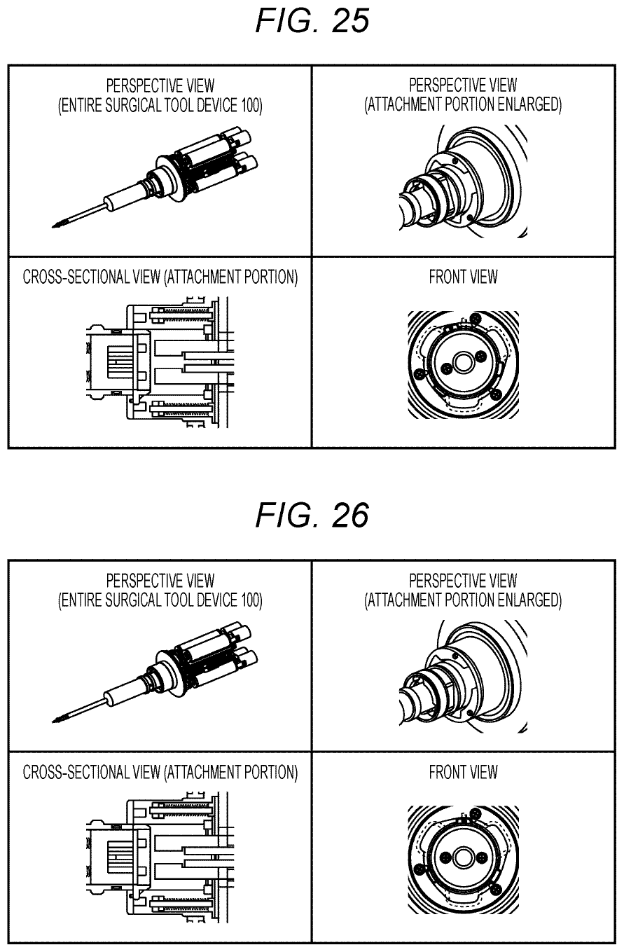

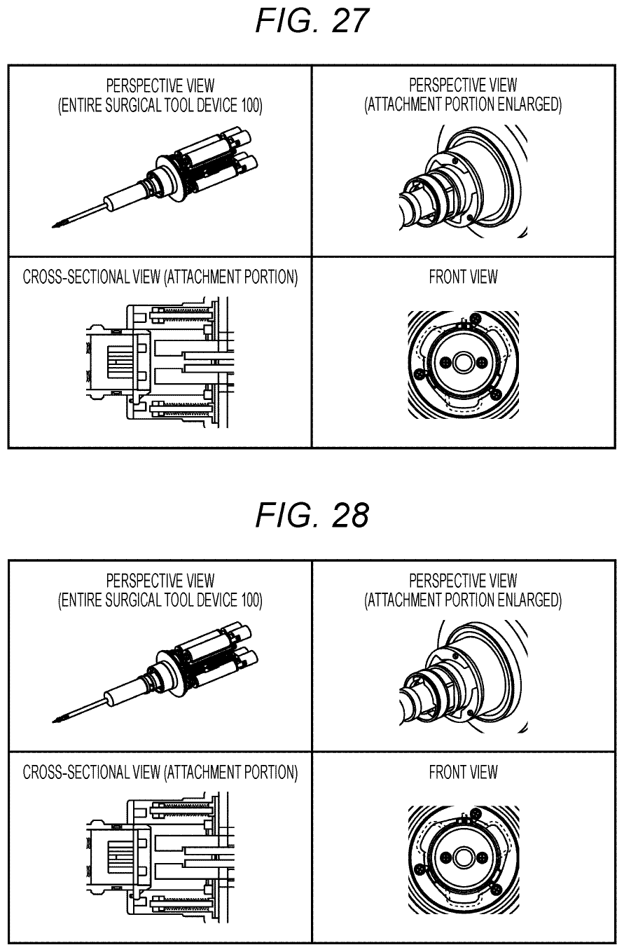

FIGS. 23 and 24 each illustrate a perspective view of the entire surgical tool device, a perspective (enlarged) view of the attachment portion, a cross-sectional view of the attachment portion, and a front view as viewed from the distal side, at a time of attachment of the surgical tool unit 101 to the adapter unit 102. Note that FIG. 23 illustrates a state before attachment, FIGS. 24 and 25 illustrate states in the middle of the attachment, and FIG. 27 illustrates a state at the time of completion of the attachment.

First, as illustrated in FIG. 23, the claws 1811 on the side of the surgical tool unit 101 are aligned with the recesses 1801A on the side of the adapter unit 102 (the front base 1801) so as to match the rotation positions.

As illustrated in FIG. 24, the claws 1811 on the side of the surgical tool unit 101 are then made to pass through the recesses 1801A on the side of the adapter unit 102 (the front base 1801), and the end portion on the root side of the surgical tool unit 101 is brought into contact with the pressing device 1803.

Next, as illustrated in FIGS. 25 and 26, the surgical tool unit 101 is rotated clockwise with respect to the adapter unit 102 toward the attachment position. As a result, the clearance between the outer diameter of the claws 1811 and the counterbore surface 1802A of the adapter base 1802 decreases. At last, the rotation positioning pin 1812 on the side of the surgical tool unit 101 is fastened to the positioning groove 1801B of the front plate 1801, the attachment positions of the surgical tool unit 101 and the adapter unit 102 are determined, and the attachment of the surgical tool unit 101 to the adapter unit 102 is completed as illustrated in FIG. 27.

### G-3. Detaching Procedures for a Surgical Tool Unit

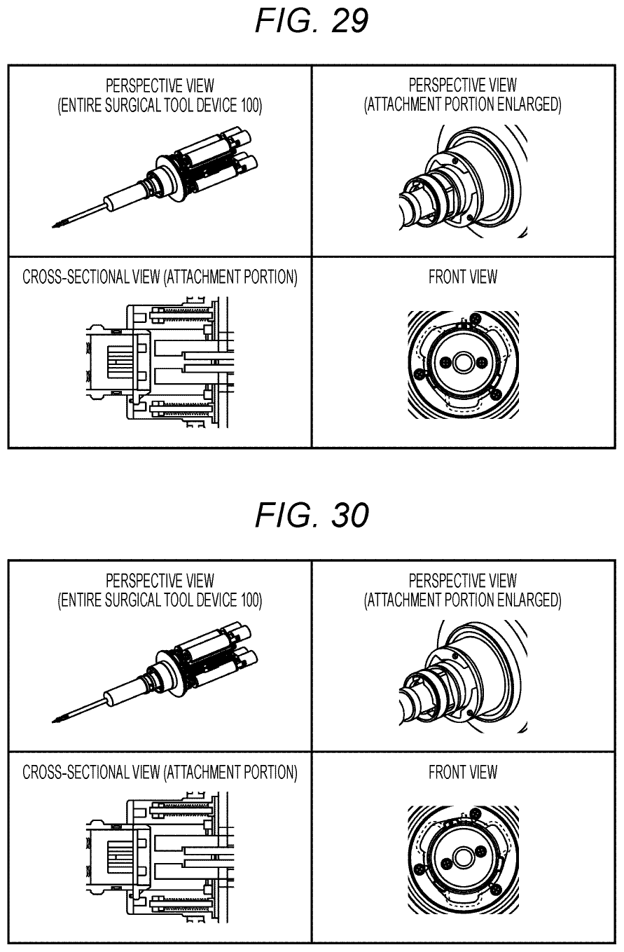

FIGS. 28 to 32 each illustrate a perspective view of the entire surgical tool device, a perspective (enlarged) view of the attachment portion, a cross-sectional view of the attachment portion, and a front view as viewed from the distal side, at a time of detachment of the surgical tool unit 101 from the adapter unit 102. Note that FIG. 28 illustrates a state before the detachment, FIGS. 29 to 31 illustrate states in the middle of the detachment, and FIG. 32 illustrates a state at the time of completion of the detachment.

The state before the detachment illustrated in FIG. 28 is the same as the state at the time of completion of the attachment illustrated in FIG. 27. First, as illustrated in FIG. 29, the surgical tool unit 101 is pushed to the proximal side until the claws 1811 of the surgical tool unit 101 come into contact with the counterbore surface 1802A of the adapter base 1802.

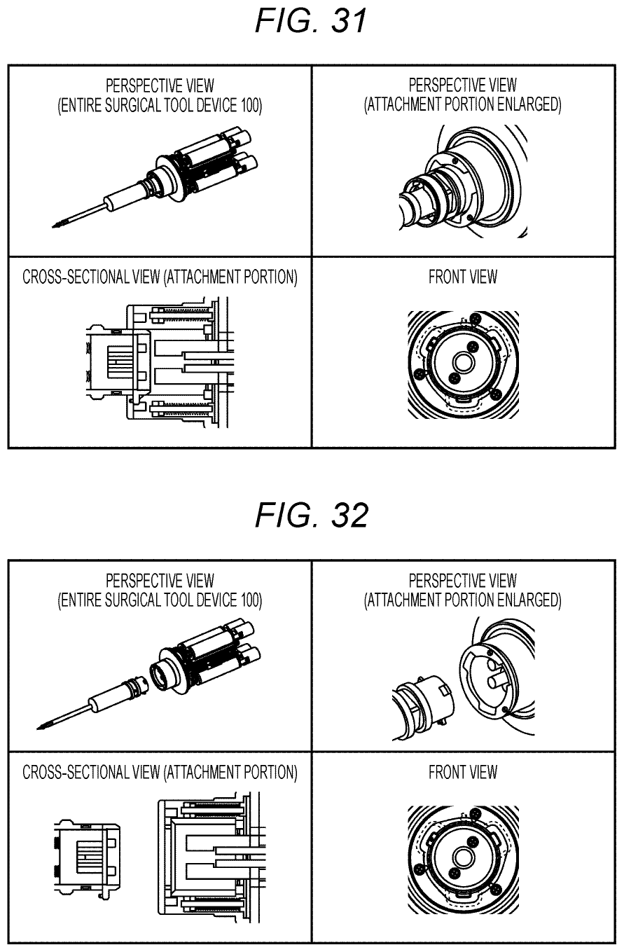

Next, as illustrated in FIG. 30, the surgical tool unit 101 is rotated counterclockwise with respect to the adapter unit 102, and the claws 1811 on the side of the surgical tool unit 101 are aligned with the recesses 1801A on the side of the adapter unit 102 (the front base 1801) to as to match the rotation positions, as illustrated in FIG. 31.

When the claws 1811 on the side of the surgical tool unit 101 come to the position where the rotation position is matched with the rotation position of the recesses 1801A on the side of the adapter unit 102 (the front base 1801), the surgical tool unit 101 is then removed from the adapter unit 102, and the detachment operation is completed as illustrated in FIG. 32.

### H. Input-Output Relationship Between a Drive Unit and a Surgical Tool Unit

The surgical tool device 100 according to the present embodiment includes the drive unit 103 including four motors and rotational-to-linear motion converting devices for the respective motors, and the surgical tool unit 101 including four cables for operating the surgical tool at the distal end and four rods to which the respective cables is connected. The surgical tool device 100 is designed to operate the surgical tool by pulling the cables with a linear motion transmission force via the rods. In this section H, the input-output relationship indicating the relationship between an input to the drive unit 103 and an output from the surgical tool unit 101 is described. Here, the input to the drive unit 103 is rotation angle command values φₘ₁ to φₘ₄ for the respective motors (however, the rotation angle is the rotation angle at the time of output after speed reduction of each motor). Further, the output from the surgical tool unit 101 is an action of the surgical tool supported at the distal end.

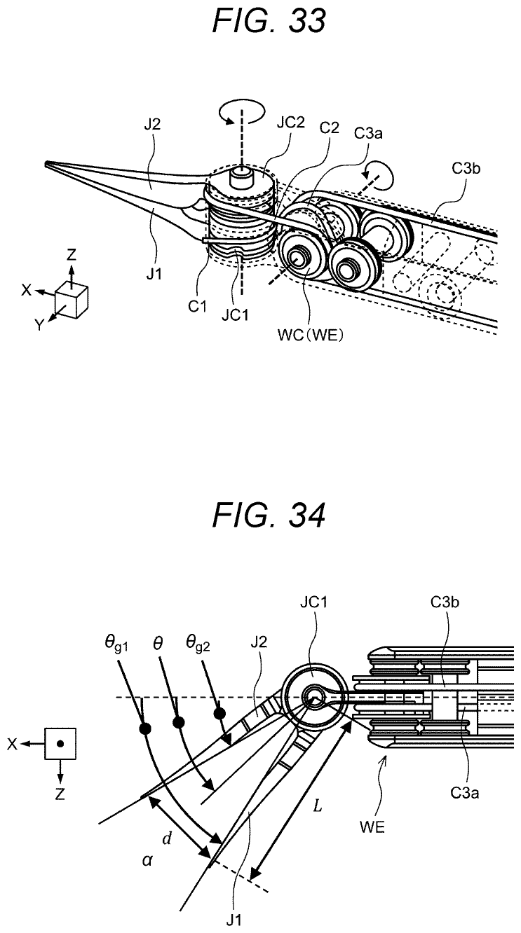

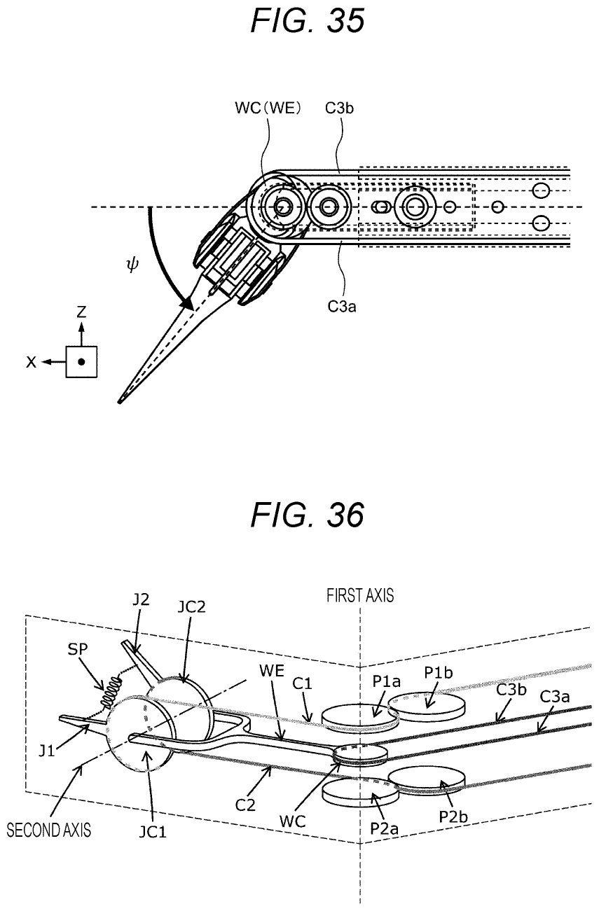

FIGS. 33 to 36 illustrate a specific configuration of a surgical tool assumed in the description of the input-output relationship. Here, the surgical tool is forceps including a pair of jaws J1 and J2 that perform an opening/closing operation. FIG. 33 is a perspective view of the forceps. FIG. 34 is a top view of the plane in which the forceps perform an opening/closing operation as viewed from above. FIG. 35 is a side view from a side surface orthogonal to the line-of-sight of FIG. 34. FIG. 36 illustrates the degree of freedom in the configuration of the forceps.

One jaw J1 is integrated with a jaw capstan JC1 having the yaw axis (second axis) as the rotation axis, and the other jaw J2 is integrated with a jaw capstan JC2 that also has the yaw axis as the rotation axis. Also, a spring (not illustrated) including a torsion spring or the like is disposed between the jaw J1 and the jaw J2 so that a repulsive force always acts in the opening direction. The jaw capstan JC1 and the jaw capstan JC2 have the same radius R_{θ}.

A cable C1 and a cable C2 are wound around the jaw capstan JC1 and the jaw capstan JC2 from directions opposite to each other. Specifically, the cable C1 is wound around the jaw capstan JC1 so as to pivot in a direction in which the jaw J1 approaches the jaw J2 when being pulled. Likewise, the cable C2 is wound around the jaw capstan JC2 so as to pivot in a direction in which the jaw J2 approaches the jaw J1 when being pulled. Accordingly, as the cable C1 and the cable C2 are pulled so that the difference in angle about the yaw axis between the jaw J1 and the jaw J2 changes, an opening/closing operation of the forceps can be performed. Also, as the cable C1 and the cable C2 are pulled so that the sum of the angles of the jaw J1 and the jaw J2 about the yaw axis changes, a turning operation of the forceps about the yaw axis can be performed.

As will be described later, the cable C1 and the cable C2 are pulled by rotative forces of a motor M1 and a motor M2 on the side of the drive unit 103, respectively. Accordingly, as the rotation angles of the motor M1 and the motor M2 are controlled, an opening/closing operation of the forceps and a turning operation of the forceps about the yaw axis can be realized.

A wrist element WE rotatably supports each of the jaw capstan JC1 and the jaw capstan JC2 about the yaw axis. Also, the wrist element WE is integrated with a wrist capstan WC having the pitch axis (first axis) as the rotation axis. As can also be seen from FIG. 36, the yaw axis (second axis) is located on the distal side, and the pitch axis (first axis) is located on the proximal side. A pair of cables C3a and C3b is wound around the wrist element WE in directions opposite to each other. Accordingly, as the pair of cables C3a and C3b is balanced, the wrist element WE, or the jaw J1 and the jaw J2 (which are the forceps) supported by the wrist element WE, can be made to turn about the pitch axis.

As will be described later, the cable C3a and the cable C3b are pulled by rotative forces of a motor M3 and a motor M4 on the side of the drive unit 103, respectively. Accordingly, as the rotation angles of the motor M3 and the motor M4 are controlled, a turning operation of the forceps about the pitch axis can be realized.

Each of the cable C1, the cable C2, the cable C3a, and the cable C3b is inserted into a shaft, is folded back to the distal side by an idler pulley, and is then connected to each corresponding rod (described above). Note that the cable C1 is inserted into the shaft, after layout adjustment is performed by an idler pulley P1a coaxial with the pitch axis, and an idler pulley P1b that is adjacent to the idler pulley P1a and has a rotation axis parallel to the pitch axis. Also, the cable C2 is inserted into the shaft, after layout adjustment is performed by an idler pulley P2a coaxial with the pitch axis, and an idler pulley P2b that is adjacent to the idler pulley P2a and has a rotation axis parallel to the pitch axis. However, the layout of the cables is not limited to the example illustrated in FIGS. 33 to 36.

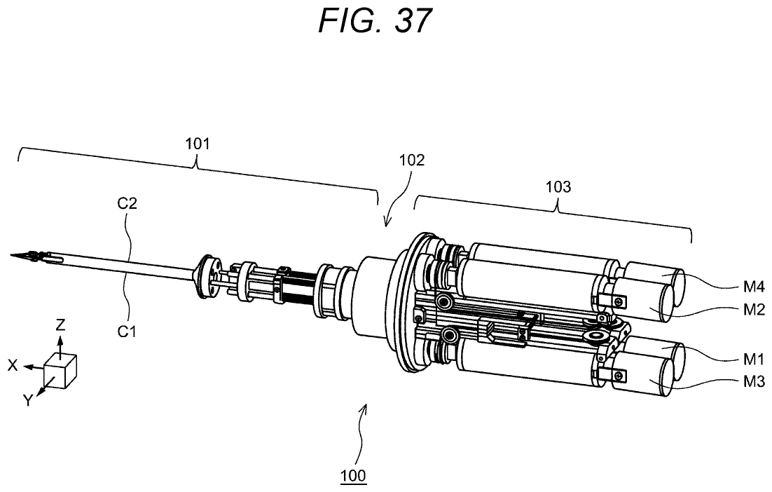

Although the side of the drive unit 103 is not illustrated in FIGS. 33 to 36, the cable C1, the cable C2, the cable C3a, and the cable C3b are pulled by the motor M1, the motor M2, the motor M3, and the motor M4 on the side of the drive unit 103 side, respectively. FIG. 37 illustrates an example of the layout of the motors M1 to M4 on the side of the drive unit 103. The rotative forces of the respective motors M1 to M4 are linearly transmitted to the side of the surgical tool unit 101 through the rotational-to-linear motion converting devices described in the above section E, and pull the cable C1, the cable C2, the cable C3a, and the cable C3b. The motor capstans attached to the output shafts of the respective motors M1 to M4 have the same radius Rm. Further, each of the motors M1 to M4 may have a planetary gear mechanism therein, but the present disclosure is not limited to this.

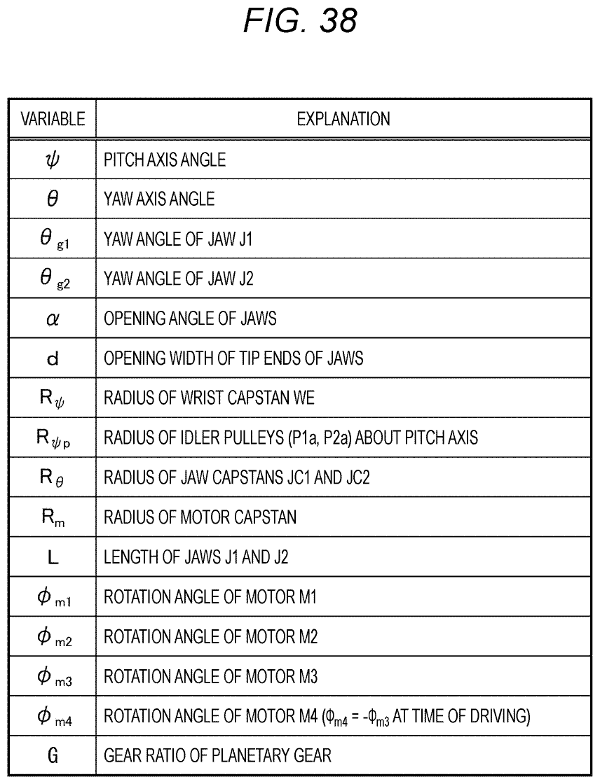

The input parameters for the drive unit 103 and the output parameters on the side of the surgical tool unit 101 are summarized in FIG. 38. In view of that, the relationship between the respective input/output parameters is expressed as in the following Expressions (1) to (6).

$$\begin{matrix}[ Math . 1 ] & \\ {θ}_{g 1} = - \frac{{R}_{m}}{G \cdot {R}_{θ}} {ϕ}_{m 1} - \frac{{R}_{ψ p}}{{R}_{θ}} ψ & ( 1 )\end{matrix}$$

$$\begin{matrix}[ Math . 2 ] & \\ {θ}_{g 2} = \frac{{R}_{m}}{G \cdot {R}_{θ}} {ϕ}_{𝔪 2} + \frac{{R}_{ψ p}}{{R}_{θ}} ψ & ( 2 )\end{matrix}$$

$$\begin{matrix}[ Math . 3 ] & \\ ψ = \frac{{R}_{m}}{G \cdot {R}_{ψ}} {ϕ}_{m 3} & ( 3 )\end{matrix}$$

$$\begin{matrix}[ Math . 4 ] & \\ θ = \frac{{θ}_{g 1} + {θ}_{g 2}}{2} & ( 4 )\end{matrix}$$

$$\begin{matrix}[ Math . 5 ] & \\ α = {θ}_{g 1} - {θ}_{g 2} & ( 5 )\end{matrix}$$

$$\begin{matrix}[ Math . 6 ] & \\ d = 2 L sin \frac{α}{2} & ( 6 )\end{matrix}$$

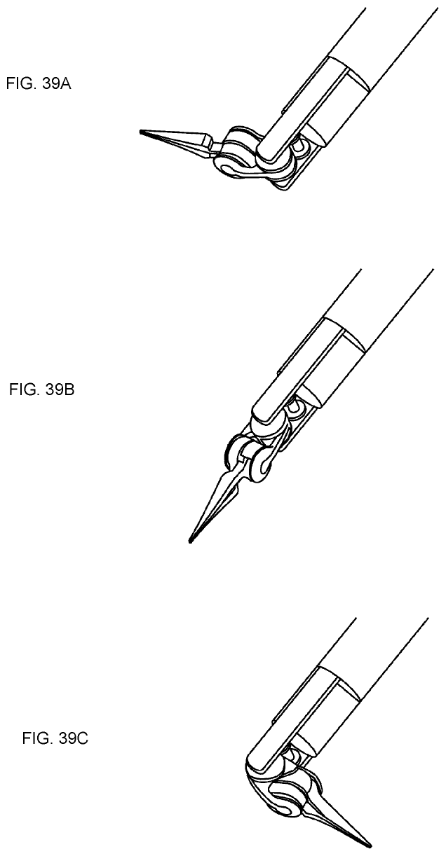

FIGS. 39(A) to 39(C) illustrate a series of operations in which the forceps at the distal end of the surgical tool unit 101 performs a yaw operation. The yaw axis angle θ of the forceps is as expressed in the above Expression (4). As described above, drive control is performed on the motor M1 and the motor M2, and the cable C1 and the cable C2 are pulled so that the sum of the angles of the jaw J1 and the jaw J2 about the yaw axis changes. Thus, a turning operation of the forceps about the yaw axis can be performed.

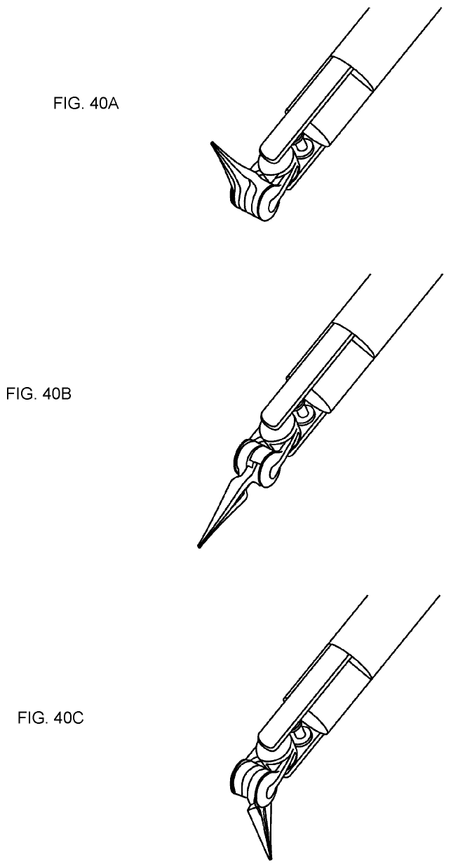

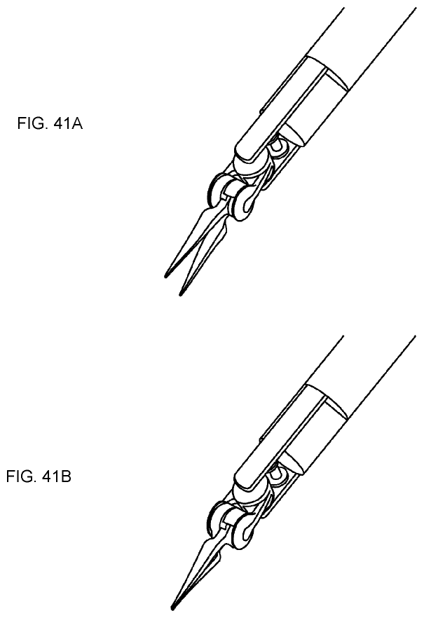

Further, FIGS. 40(A) to 40(C) illustrate a series of operations in which the forceps at the distal end of the surgical tool unit 101 perform a pitch operation. The pitch axis angle ψ of the forceps is as expressed in the above Expression (3). As described above, drive control is performed on the motor M3 and the motor M4, and the cable C3a and the cable C3b are pulled so as to be balanced. Thus, a turning operation of the forceps about the pitch axis can be performed.

Furthermore, FIGS. 41(A) and 41(B) illustrate a series of operations in which the forceps at the distal end of the surgical tool unit 101 perform an opening/closing operation. The opening angles α of the jaw J1 and the jaw J2, and the opening widths of the tip ends of the jaw J1 and the jaw J2 are as expressed in the above Expressions (5) and (6), respectively. As described above, drive control is performed on the motor M1 and the motor M2, and the cable C1 and the cable C2 are pulled so that the difference in the angle about the yaw axis between the jaw J1 and the jaw J2 changes. Thus, an opening/closing operation of the forceps can be performed.

### I. Arm Device

In the above section B, an aspect in which the surgical tool device 100 according to the present disclosure is mounted on the arm device 300, and the surgical tool unit 101 is made to perform a pan operation and a tilt operation has been described with reference to FIG. 3. In this section I, a specific configuration of the arm device 300 and operations of the surgical tool unit 101 mounted on the arm device 300 are described.

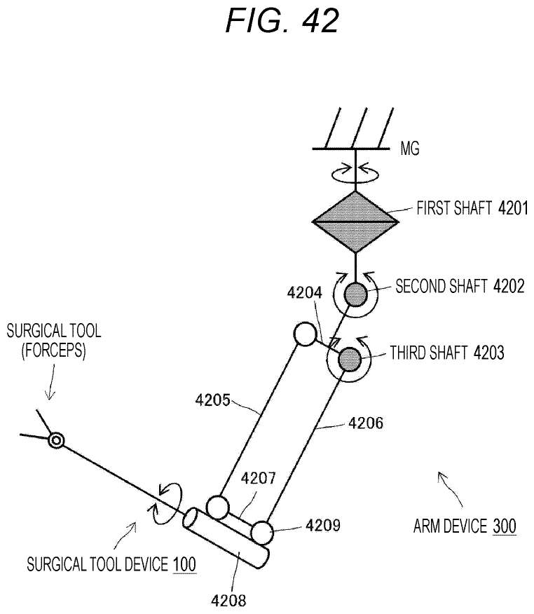

FIG. 42 illustrates an example of the degree of freedom in the configuration of the arm device 300 illustrated in FIG. 3. For ease of explanation, it is assumed that the arm device 300 is suspended from a ceiling that is a mechanical ground (MG). In FIG. 42, only the active joints among the joint shafts of the arm device 300 are colored in gray.

The arm device 300 includes, in order from the top, a first shaft 4201 that rotates about a vertical pan axis, a second shaft 4202 that rotates about a horizontal tilt axis, and a four-joint link mechanism including four links 4204 to 4207. The first shaft 4201 and the second shaft 4202 are active shafts.

Among the joints included in the four-joint link mechanism, a third shaft 4203 is an active shaft, and the other joints are passive shafts. Therefore, the four-joint link mechanism includes a prime mover link 4204 driven by the third shaft 4203, two intermediate links 4205 and 4206, and a driven link 4207 that operates following the prime mover link 4204 via the intermediate links 4205 and 4206. Further, the surgical tool device 100 is supported by a device holder 4208 integrated with the driven link 4207. As described above, the surgical tool device 100 includes the surgical tool unit 101, the adapter unit 102, and the drive unit 103, which are not specifically illustrated in FIG. 32 though. The device holder 4208 includes a mechanism that rotates the surgical tool device 100 about the longitudinal axis of the shaft 102, which is not specifically illustrated in the drawing though.

The first shaft 4201 realizes a pan operation to rotate the entire arm device 300 about the vertical pan axis with respect to the mechanical ground. Further, the second shaft 4203 couples the output shaft of the first shaft to the four-joint link mechanism, and realizes a first tilt operation of rotating the entire four-joint link mechanism about the tilt axis. Furthermore, the third shaft 4203 can rotate the prime mover link 4204 about the third shaft 4203 to cause the driven link 4207 to follow the rotation in the four-joint link mechanism. As a result, the third shaft 4203 realizes a second tilt operation of rotating the surgical tool device 100 supported by the device holder 4208 integrated with the driven link 4207 about a joint shaft 4209 at the lowermost end.

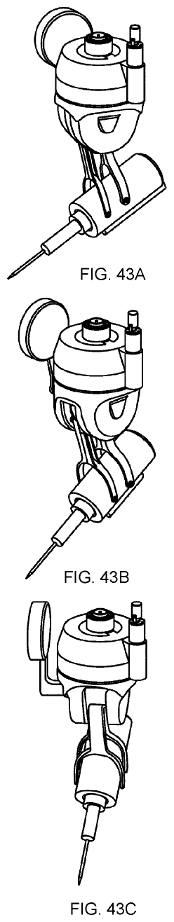

FIGS. 43(A) to 43(C) illustrate a series of operations in which the arm device 300 pans the surgical tool device 100. The arm device 300 can cause the surgical tool device 100 to perform a pan operation about the first shaft 3201 by driving the first shaft 3201.

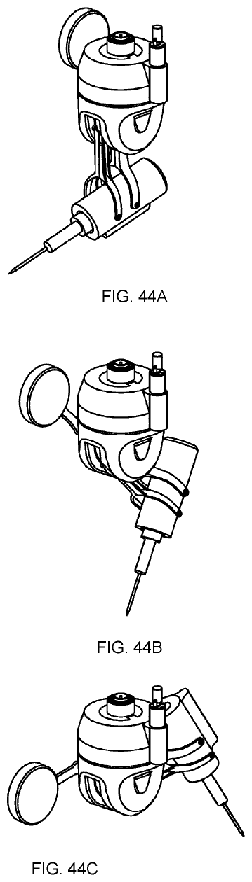

Further, FIGS. 44(A) to 44(C) illustrate a series of operations in which the arm device 300 causes the surgical tool device 100 to tilt with respect to the main unit of the arm device 300. When the second shaft 4203 rotates, the entire four-joint link mechanism is rotated about the tilt axis, and the surgical tool device 100 can perform a tilt operation about the second shaft 4203 (this is referred to as the “first tilt operation”).

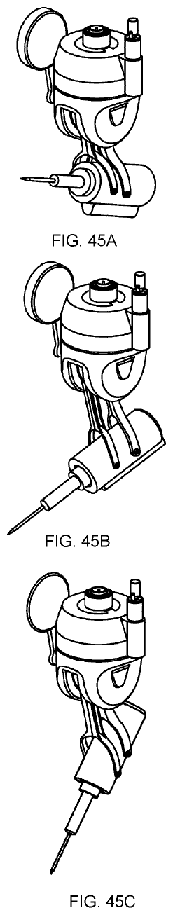

Furthermore, FIGS. 45(A) to 45(C) illustrate a series of operations in which the arm device 300 causes the surgical tool device 100 to tilt at the current position. When the third shaft 4203 rotates, the prime mover link 4204 rotates about the third shaft 4203, and the driven link 4207 rotates about the joint shaft 4209 so as to follow the prime mover link 4204. As a result, the surgical tool device 100 supported by the device holder 4208 integrated with the driven link 4207 can be rotated about the joint shaft 3209 at the lowermost end (this is referred to as the “second tilt operation”).

### J. Master-Slave System

In general, surgical operation is a difficult task that is performed by the operator's sensorimotor. Recently, a master-slave surgical system has been introduced to suppress tremor of the operator and realize precise surgery. The arm device 300 described in the above section I can be applied to a master-slave system as a slave robot that is remotely controlled from the master side.

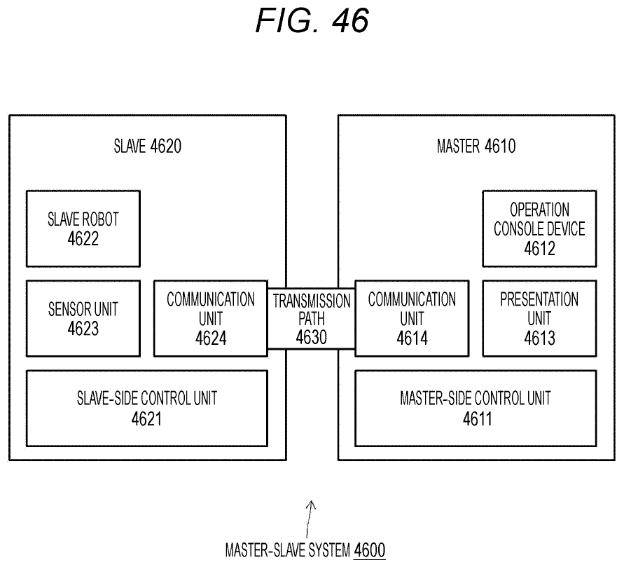

FIG. 46 schematically illustrates an example of the functional configuration of a master-slave system 4600. The master-slave system 4600 illustrated in the drawing includes a master 4610 that remotely controls a slave robot, and a slave 4620 including the slave robot. In a case where the master-slave system 4600 is applied to surgery, a user such as a surgeon operates an operation console device on the side of the master 4610, and driving of a slave robot 4622 formed with the arm device 300 and the like is controlled in accordance with the operation by the user on the side of the slave 4620 installed in the operating room, so that the surgery can be performed.

The master 4610 is installed outside the operating room (alternatively, a place separated from the operating table in the operating room), for example, and the user (operator) remotely operates the slave 4620. The slave 4620 includes the slave robot 4622 such as the arm device 300 installed near the operating table. The arm device 300 supports the surgical tool device 100 as an end effector including a surgical tool and an observation device, and realizes a pan operation, a tilt operation, and the like as described in the above section I. The surgical tool herein is a medical instrument such as forceps, a pneumoperitoneum tube, an energy treatment tool, tweezers, or a retractor, for example, and the observation device is an endoscope, for example. The slave robot 4622 then performs surgery for a patient laid on the operating table in accordance with an instruction from the master 4610. Examples of the surgery described herein include a laparoscopic surgery, a celoscopic surgery, a brain surface surgery, and an eyeball or eyeground surgery, for example. The master 4610 and the slave 4620 are interconnected via a transmission path 4630. The transmission path 4630 is desirably capable of performing signal transmission with a low delay using a medium such as an optical fiber, for example.

The master 4610 includes a master-side control unit 4611, an operation console device 4622, a presentation unit 4613, and a master-side communication unit 4614. The master 4610 operates under the overall control of the master-side control unit 4611.

The operation console device 4622 is an input device for the user (the operator or the like) to perform a remote operation or an on-screen 3D operation for the slave robot 4622 that has a surgical tool such as forceps mounted thereon in the slave 4620. It is assumed that the operation console device 4622 can perform operations of three degrees of freedom in translation for translating the surgical tool, three degrees of freedom in rotation for changing a posture of the surgical tool, and one degree of freedom in gripping such as an opening/closing operation of the forceps, for example.

The presentation unit 4613 presents information regarding surgery being performed in the slave 4620 to the user (operator) operating the operation console device 4622, on the basis of sensor information mainly acquired by a sensor unit 4623 (described later) on the side of the slave 4620.

For example, in a case where the sensor unit 4623 on the side of the slave 4620 is equipped with an RGB camera for observing the surface of the affected area, an RGB camera for capturing a microscopic image, an endoscope in laparoscopic or celoscopic surgery, or an interface for capturing captured images of these cameras, and image data of these devices are transferred to the operation console device 4612 with a low delay through the transmission path 4630, the presentation unit 4613 displays a captured image of the affected site of the affected area in real time on a screen, using a monitor display or the like.

Further, in a case where the sensor unit 4623 is equipped with a function to measure a force such as an external force or a moment acting on the surgical tool mounted on the slave robot 4622, and such haptic information is transferred to the master 4610 with a low delay via the transmission path 4630, the presentation unit 4613 performs haptic presentation to the user (operator). The haptic presentation function of the presentation unit 4613 is incorporated and implemented in the operation console device 4622. Specifically, the presentation unit 4613 performs haptic presentation to the user (operator) by driving a grip portion having three degrees of freedom in rotation and one degree of freedom in holding of the tip end of the operation console device 4622 with a motor, for example.

The master-side communication unit 4614 performs a signal transmission/reception process with the slave 4620 via the transmission path 4630, under the control of the master-side control unit 4611. For example, in a case where the transmission path 4630 includes an optical fiber, the master-side communication unit 4614 includes an electro-optical conversion unit that converts an electrical signal transmitted from the master 4610 into an optical signal, and a photoelectric conversion unit that converts an optical signal received from the transmission path 4630 into an electrical signal. The master-side communication unit 4614 transfers an operation command for the slave robot 4622 input by the user (operator) via the master 4610, to the slave 4620 via the transmission path 4630. Furthermore, the master-side communication unit 4614 receives the sensor information transmitted from the slave 4620 via the transmission path 4630.

Meanwhile, the slave 4620 includes a slave-side control unit 4621, the slave robot 4622, the sensor unit 4623, and a slave-side communication unit 4624. The slave 4620 operates in accordance with an instruction from the master 4610, under the overall control of the slave-side control unit 4621.

The slave robot 4622 is an arm-type surgical robot having an articulated link structure such as the above-described arm device 300, for example, and is equipped with a surgical tool as an end effector and an observation device at the tip end (or the distal end). Examples of the surgical tool include forceps, a pneumoperitoneum tube, an energy treatment tool, tweezers, and a retractor. Further, examples of the observation device include an endoscope and the like. The slave-side control unit 4621 interprets an operation command transmitted from the master 4610 via the transmission path 4630, converts the operation command into a drive signal for an actuator that drives the slave robot 4622, and outputs the drive signal. The slave robot 4622 then operates on the basis of the drive signal from the slave-side control unit 4621.

The sensor unit 4623 includes a plurality of sensors for detecting the status of the slave robot 4622 and the status of the affected area in the operation being performed by the slave robot 4622, and further includes an interface for taking in sensor information from various sensor devices installed in the operating room. For example, the sensor unit 4623 includes a force torque sensor (FTS) for measuring an external force and a moment acting during the operation on the surgical tool mounted on the tip end (distal end) of the slave robot 4622. Further, the sensor unit 4623 is equipped with an observation device such as an RGB camera for observing the surface of the affected area during surgery by the slave robot 4622, an RGB camera for capturing a microscopic image, or an endoscope in laparoscopic or celoscopic surgery, or is equipped with an interface for taking in images captured by these cameras.

The slave-side communication unit 4624 performs a signal transmission/reception process with the master 4610 via the transmission path 4630, under the control of the slave-side control unit 4621. For example, in a case where the transmission path 4630 includes an optical fiber, the slave-side communication unit 4624 includes an electro-optical conversion unit that converts an electrical signal transmitted from the slave 4620 into an optical signal, and a photoelectric conversion unit that converts an optical signal received from the transmission path 4630 into an electrical signal.

The slave-side communication unit 4624 transfers the haptic data of the surgical tool acquired by the sensor unit 4623, and images captured by an RGB camera for observing the surface of the affected area, an RGB camera for capturing a microscopic image, and an endoscope or the like in laparoscopic or celoscopic surgery, to the operation console device 4612 through the transmission path 4630. Further, the slave-side communication unit 4624 receives, through the transmission path 4630, an operation command for a surgical manipulator 122 transmitted from the master 4610.

On the side of the master 4610, an operation command for remotely operating the slave robot 4622 is input via the operation console device 4612. The operation command includes a pan operation of the arm device 300 (see FIG. 43), the first tilt operation of the arm device 300 (see FIG. 44), the second tilt operation of the arm device 300 (see FIG. 45), a rotation operation about the longitudinal axis (or roll axis) of the surgical tool device 100 held by the arm device 300, and an operation of the surgical tool at the distal end of the surgical tool unit 101.

The slave-side control unit 4621 performs drive control on the active shafts (the first to third shafts) of the arm device 300 and drive control on the drive device 103, so as to realize operations of the arm device 300 and the surgical tool unit 101 in accordance with the received operation command.

On the side of the master 4610, instructions are issued via the operation console device 4612, regarding an operation of the arm device 300 (see FIGS. 39 to 41), an operation of a surgical tool (specifically, a yaw operation of forceps (see FIG. 39), a pitch operation of the forceps (see FIG. 40), and an opening/closing operation of the forceps (see FIG. 41). On the other hand, in a case where an operation command designating the yaw axis angle θ of the forceps, the pitch axis angle of the forceps, and the opening angle α or the opening width d of the forceps is received from the side of the master 4610, the slave-side control unit 4621 calculates the rotation angle of each of the motors M1 to M4 in the drive unit 103 for realizing the instructed operation of the forceps, and generates an angle command to each of the motors M1 to M4.

The input-output relationship between the drive unit 103 and the surgical tool unit 101 is as described in the above section H. Since the drive unit 103 to which the present embodiment is applied operates without any backlash but has a high backdrivability, the forceps of the surgical tool unit 101 can be accurately driven on the basis of the angle command to each of the motors M1 to M4.

Further, in a case where the forceps receives an external force from the outside (such as the affected area) during surgery of being performed by the slave robot 4622, and the jaw J1 or J2 is displaced, displacement angles Δφₘ₁ to Δφₘ₄ of the respective motors are calculated on the basis of a result of detection by the encoders of the motors M1 to M4, are converted into displacement amounts (Δθ_{g1}, Δθ_{g1}, Δα, and Δd) of the jaw J1 or J2, and are sent as haptic feedback information to the side of the master 4610. Since the drive unit 103 to which the present embodiment is applied has a high backdrivability, the amount of displacement of the surgical tool can be measured with high accuracy on the basis of the displacement angle of the motor. Thus, accurate haptic feedback information can be supplied to the side of the master 4610 to realize precise surgery.

### INDUSTRIAL APPLICABILITY

The present disclosure has been described in detail with reference to a specific embodiment. However, it is obvious that those skilled in the art can make modifications and substitutions of the embodiment without departing from the scope of the present disclosure.

In the present specification, the embodiment in which the present disclosure is mainly applied to surgery in the medical field has been mainly described, but the gist of the present disclosure is not limited to this. The present disclosure can be applied to a wide variety of fields such as a remote operation robot that performs precise work in a difficult-to-work space such as a manufacturing factory, a construction site, or outer space, and an operation console device for remote operation, and can reduce the sizes and the weights of the drive unit and the entire device. Furthermore, an end effector unit not including any rotational-to-linear motion converting device can be made smaller in size and diameter. Accordingly, it is easy to install a plurality of types of end effector units on a mounting table (a surgical tool stand or the like) in the vicinity of a robot, and the robot can automatically change end effector units in a short time.

In short, the present disclosure has been described in an illustrative manner, and the contents disclosed in the present specification should not be interpreted in a limited manner. To determine the subject matter of the present disclosure, the claims should be taken into consideration.

Note that the present disclosure may also have the following configurations.

(1) A drive device including:
a capstan;
a cable wound around the capstan; and
a rod that has one degree of freedom in linear motion, and performs a linear motion by rotation of the capstan, the cable being connected to the rod,
in which a replaceably attached end effector is driven by the linear motion of the rod.

(2) The drive device according to (1), in which
the cable includes a pair of cables that are wound around the capstan in directions opposite to each other and are connected to the rod in forward and backward directions, and
the rod moves forward and backward in accordance with a direction of rotation of the capstan.

(3) The drive device according to (2), in which
the capstan includes a capstan assembly including a first capstan and a second capstan that are coaxially arranged, and
the pair of cables is wound around each of the first capstan and the second capstan in directions opposite to each other.

(4) The drive device according to (3), in which
the capstan assembly includes a reaction force applying unit that applies a reaction force for rotating in directions opposite to each other between the first capstan and the second capstan.

(5) The drive device according to (4), in which
the pair of cables is wound around the first capstan and the second capstan by rotation caused by the reaction force, and a pre-tension force is applied to the pair of cables.

(6) The drive device according to (4) or (5), in which
the reaction force applying unit includes a torsion spring or another elastic member that is disposed between the first capstan and the second capstan.

(7) The drive device according to any one of (1) to (6), in which
the capstan is attached to at least one of an input shaft or an output shaft for power.

(8) The drive device according to any one of (1) to (7), further including
a motor in which the capstan is attached to at least one of an input shaft or an output shaft.

(9) The drive device according to (8), in which
the rod is secured to a slide base that is slidably guided in the one direction via a linear guide with respect to a unit base on which the motor is mounted.

(10) A surgical tool device including:
a drive unit including: a motor; a capstan attached to an input shaft or an output shaft of the motor; a cable wound around the capstan; and a rod that linearly moves by rotation of the motor, the cable being connected to the rod; and
a surgical tool unit that is replaceably attached to the drive unit, and drives a surgical tool with a linear motion force transmitted via the rod.

(11) An arm device including:
a surgical tool device including: a drive unit including: a motor; a capstan attached to an input shaft or an output shaft of the motor; a cable wound around the capstan; and a rod that linearly moves by rotation of the motor, the cable being connected to the rod; and a surgical tool unit that is replaceably attached to the drive unit, and drives a surgical tool with a linear motion force transmitted via the rod; and
an arm of an articulated link structure that supports the surgical tool device.

(12) A master-slave system including:
a slave device including: a surgical tool device including: a drive unit including: a motor; a capstan attached to an input shaft or an output shaft of the motor; a cable wound around the capstan; and a rod that linearly moves by rotation of the motor, the cable being connected to the rod; and a surgical tool unit that is replaceably attached to the drive unit, and drives a surgical tool with a linear motion force transmitted via the rod; and an arm of an articulated link structure that supports the surgical tool device; and
a master device that operates the surgical tool device and the arm.

### REFERENCE SIGNS LIST

101 Surgical tool unit
102 Adapter unit
103 Drive unit
300 Arm device
401 Surgical tool
402 Shaft
403 Surgical tool unit base
411, 421 Cable
412, 422 Pulley
413, 423 Rod
414, 424 Spring
501 Drive unit base
511, 521 Motor
512, 522 Speed reducer
513, 523 Encoder
514, 524 Motor capstan
515, 516, 525, 526 Cable
515A, 516A, 525A, 526A Idler pulley
517, 527 Rod
518, 528 Linear guide
701 Surgical tool
702 Shaft
703 Inner base
704 Joining portion
705 Case
711, 721 Cable
712, 722 Pulley
713, 723 Rod
714 Rotation inhibiting device
714A Protrusion
715 Spring
811, 812 Bearing (plain bearing)
910 Shaft connecting portion
920 Rod support portion
921 to 924 Rod insertion hole
930 Frame
931 Guide groove
940 Pulley support portion
1401 Printed wiring board
1501 Drive unit base
1511 Motor
1512 Speed reducer
1513 Encoder
1521, 1522 Motor capstan
1523, 1524 Cable
1525 Rod
1526 Slide base
1528 Cable securing portion
1531 to 1533 Idler pulley
1801 Front plate
1801A Recess
1801B Positioning groove
1802 Adapter base
1802A Counterbore surface
1803 Pressing device
1804 Spring
1805 Spring securing plate
1806 Guide shaft
1811 Claw
1812 Positioning pin
4600 Master-slave system
4610 Master
4611 Master-side control unit
4612 Operation console device
4613 Presentation unit
4614 Master-side communication unit
4620 Slave
4621 Slave-side control unit
4622 Slave robot
4623 Sensor unit
4624 Slave-side communication unit
4630 Transmission path

The invention claimed is:

1. A drive device comprising:
a capstan;
a cable wound around the capstan; and
a rod that has one degree of freedom in linear motion, and performs a linear motion by rotation of the capstan, the cable being connected to the rod,
wherein a replaceably attached end effector is driven by the linear motion of the rod.

2. The drive device according to claim 1, wherein
the cable includes a pair of cables that are wound around the capstan in directions opposite to each other and are connected to the rod in forward and backward directions, and
the rod moves forward and backward in accordance with a direction of rotation of the capstan.

3. The drive device according to claim 2, wherein
the capstan includes a capstan assembly including a first capstan and a second capstan that are coaxially arranged, and
the pair of cables is wound around each of the first capstan and the second capstan in directions opposite to each other.

4. The drive device according to claim 3, wherein
the capstan assembly includes a reaction force applying unit that applies a reaction force for rotating in directions opposite to each other between the first capstan and the second capstan.

5. The drive device according to claim 4, wherein
the pair of cables is wound around the first capstan and the second capstan by rotation caused by the reaction force, and a pre-tension force is applied to the pair of cables.

6. The drive device according to claim 4, wherein
the reaction force applying unit includes a torsion spring or another elastic member that is disposed between the first capstan and the second capstan.

7. The drive device according to claim 1, wherein
the capstan is attached to at least one of an input shaft or an output shaft for power.

8. The drive device according to claim 1, further comprising
a motor in which the capstan is attached to at least one of an input shaft or an output shaft.

9. The drive device according to (8), in which
the rod is secured to a slide base that is slidably guided in the one direction via a linear guide with respect to a unit base on which the motor is mounted.

10. A surgical tool device comprising:
a drive unit including: a motor; a capstan attached to an input shaft or an output shaft of the motor; a cable wound around the capstan; and a rod that linearly moves by rotation of the motor, the cable being connected to the rod; and
a surgical tool unit that is replaceably attached to the drive unit, and drives a surgical tool with a linear motion force transmitted via the rod.

11. An arm device comprising:
a surgical tool device including: a drive unit including: a motor; a capstan attached to an input shaft or an output shaft of the motor; a cable wound around the capstan; and a rod that linearly moves by rotation of the motor, the cable being connected to the rod; and a surgical tool unit that is replaceably attached to the drive unit, and drives a surgical tool with a linear motion force transmitted via the rod; and
an arm of an articulated link structure that supports the surgical tool device.

12. A master-slave system comprising:
a slave device including: a surgical tool device including: a drive unit including: a motor; a capstan attached to an input shaft or an output shaft of the motor; a cable wound around the capstan; and a rod that linearly moves by rotation of the motor, the cable being connected to the rod; and a surgical tool unit that is replaceably attached to the drive unit, and drives a surgical tool with a linear motion force transmitted via the rod; and an arm of an articulated link structure that supports the surgical tool device; and
a master device that operates the surgical tool device and the arm.
